(19) United States
(12) Patent Application Publication
(10) Pub. No.: US 2010/0278767 A1
(43) Pub. Date: Nov. 4, 2010

(54) Lightening Agents and/or Dyes that Contain Aldehyde(s)

(75) Inventors: Horst Höffkes, Dusseldorf (DE); Denise Fuhr, Hamburg (DE); Wibke Gross, Huckelhoven (DE); Georg Knübel, Dusseldorf (DE); Doris Oberkobusch, Dusseldorf (DE); Anja Reichert, Dusseldorf (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Dusseldorf (DE)

(21) Appl. No.: 12/377,395

(22) PCT Filed: Aug. 9, 2007
(86) PCT No.: PCT/EP07/58269

§ 371 Date: Mar. 31, 2009

(30) Foreign Application Priority Data
Aug. 15, 2006 (DE) 102006038343.5

(51) Int. Cl. A61K 8/33; A61Q 5/08; A61Q 5/00

(52) U.S. Cl. 424/62

(57) ABSTRACT

Agents for dyeing and/or lightening keratin fibers, in particular human hair, containing, relative to the weight thereof, 0.001 to 15 wt. % of at least one aldehyde of the formula (I):

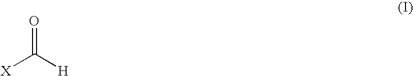

wherein X represents —CH(R²)—SO₂—Y—R¹, —CR³R⁴R⁵, or

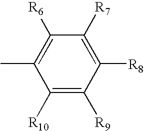

wherein Y represents —CH(CHO)— or —CH₂— or a chemical bond, and
wherein each of R¹, R², R³, R⁴, R⁵, R⁶, R⁷, R⁸, R⁹, and R¹⁰ independently represents —H or —CN or —F or —Cl or —Br or —I or —CHO or —NH₂ or —NO₂ or —CF₃ or —CCl₃ or —CF₂CF₃ or —CCl₂CCl₃ or an optionally substituted (C₁-C₆) alkyl group or a hydroxyalkyl group or a polyhydroxyalkyl group or an optionally substituted (C₁-C₆) alkylene group, and wherein the agent contains no oxidation dye precursors of developer and coupler type.

The present invention relates to agents for dyeing and/or lightening keratin fibers, i.e. agents for application onto keratin fibers, in particular human hair, and to the use thereof.

Modifying the shape and color of hair is an important area of modern cosmetics. In this way, the hair's appearance can be adapted both to current fashion trends and to a person's individual wishes. Permanent waves and other methods for modifying hair shape may be applied virtually irrespective of the type of hair to be treated. In contrast, dyeing and blonding methods are restricted to specific initial hair colors. The principles of blending methods are known to a person skilled in the art and may be looked up in relevant monographs, for example by Kh. Schräder, Grundlagen and Rezepturen der Kosmetika, 2nd edition, 1989, Dr. Alfred Hüthig Verlag, Heidelberg, or W. Limbach (ed.), Kosmetik, 2nd edition, 1995, Georg Thieme Verlag, Stuttgart, N.Y.

It is, however, often simultaneously desired to change hair color and not only to dye the hair but also to lighten the hair to be dyed.

Conventional hair dyes generally consist of at least one developer substance and at least one coupler substance and they optionally also contain direct dyes as tinting agents. The coupler and developer components are also known as oxidation dye precursors.

Before application onto human hair, hair dyes and/or lightening agents in solid or pasty form are mixed with a dilute aqueous hydrogen peroxide solution. This mixture is then applied onto the hair and rinsed off again after a specific exposure time. The duration of the exposure time on the hair in order to achieve complete decolorization or lightening is between approx. 30 and 40 minutes. Obviously, there is a requirement among users of these hair dyes or blonding agents to shorten this exposure time.

Currently commercially available dyes and/or blonding agents, whether in paste or powder form, cannot be considered ideal. While the dyeing and/or blonding action on the hair can be regarded as tailored to the user's requirements, a series of disadvantages and problems remain during both production and handling of these agents.

Coloring and blonding processes on keratin fibers accordingly conventionally proceed at alkaline pH values, in particular between 9.0 and 10.5. These pH values are necessary to ensure that the external cuticle opens up and the active species (dyes or precursors and/or hydrogen peroxide) penetrate into the hair. The alkalizing agent which is conventionally used is ammonia, but this has the disadvantage for the user of an intense odor and possible irritation.

Aminomethylpropanol or monoethanolamine are, for example, used in commercial products as alternative alkalizing agents to ammonia. However, since they often perform less well than ammonia, they are conventionally used in a blend with ammonia, but this also impairs the performance of the ammonia, in particular with regard to the parameters gray coverage, lightening power and dyeing result.

A need accordingly still remains for active ingredients or active ingredient combinations for lightening agents and dyes which have a reduced ammonia content. In particular, the intention is to obtain ammonia-reduced or even ammonia-free formulations containing dyes and/or hydrogen peroxide which have performance comparable with that of conventional ammonia-based agents.

It has now been found that aldehydes may particularly advantageously be incorporated into hair dyes and lightening agents and enhance the performance of these agents.

In a first embodiment, the present invention provides agents for dyeing and/or lightening keratin fibers, in particular human hair, containing, relative to the weight thereof, 0.001 to 15 wt. % of at least one aldehyde of the formula (I):

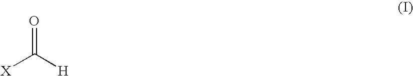

in which
X denotes —CH(R²)—SO₂—Y—R¹, —CR³R⁴R⁵,

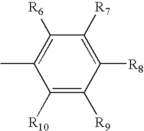

Y denotes —CH(CHO)— or —CH₂— or a chemical bond
R¹, R², R³, R⁴, R⁵, R⁶, R⁷, R⁸, R⁹, R¹⁰ mutually independently denote —H or —CN or —F or —Cl, or —Br, or —I or —CHO or —NH₂ or —NO₂ or —CF₃ or —CCl₃ or —CF₂CF₃ or —CCl₂CCl₃ or an optionally substituted (C₁-C₆) alkyl group or a hydroxyalkyl group or a polyhydroxyalkyl group or an optionally substituted (C₁-C₆) alkylene group,
   wherein the agent contains no oxidation dye precursors of the developer and coupler type.

Keratin fibers should here be taken to mean furs, wool, feathers and in particular human hair. Although the agents according to the invention are primarily suitable for dyeing and/or lightening keratin fibers, there is no reason in principle why they should not also be used in other fields.

The agents according to the invention contain at least one aldehyde of the formula (I) as an essential constituent. Among these, some representatives are preferred. In particular, X preferably denotes —CH₂—SO₂—H, —CH(CH₃)—SO₂—H, —CH(CH₂CH₃)—SO₂—H, —CH((CH₂)₂CH₃)—SO₂—H, —CH(CH(CH₃)₂)—SO₂—H, —CH(CN)—SO₂—H, —CH(CHO)—SO₂—H, —CH(NH₂)—SO₂—H, —CH(NH₂)—SO₂—H, —CH(NO₂)—SO₂—H, —CH(F)—SO₂—H, —CH(Cl)—SO₂—H, —CH(Br)—SO₂—H, —CH(I)—SO₂—H, —CH₂—SO₂—CH₂—H, —CH(CH₃)—SO₂—CH₂—H, —CH(CH₂CH₃)—SO₂—CH₂—H, —CH((CH₂)₂CH₃)—SO₂—CH₂—H, —CH(CH(CH₃)₂)—SO₂—CH₂—H, —CH(CN)—SO₂—CH₂—H, —CH(CHO)—SO₂—CH₂—H, —CH(NH₂)—SO₂—CH₂—H, —CH(NO₂)—SO₂—CH₂—H, —CH(F)—SO₂—CH₂—H, —CH(Cl)—SO₂—CH₂—H, —CH(Br)—SO₂—CH₂—H, —CH(I)—SO₂—CH₂—H, —CH₂—SO₂—CH(CHO)—H, —CH(CH₃)—SO₂—CH(CHO)—H, —CH(CH₂CH₃)—SO₂—CH(CHO)—H, —CH((CH₂)₂CH₃)—SO₂—CH(CHO)—H, —CH(CH(CH₃)₂)—SO₂—CH(CHO)—H, —CH(CN)—SO₂—CH(CHO)—H, —CH(CHO)—SO₂—CH(CHO)—H, —CH(NH₂)—SO₂—CH(CHO)—H, —CH(NO₂)—SO₂—CH(CHO)—H, —CH(F)—SO₂—CH(CHO)—H, —CH(Cl)—SO₂—CH(CHO)—H, —CH(Br)—SO₂—CH(CHO)—H, —CH(I)—SO₂—CH(CHO)—H, —CH₂—SO₂—CH₃, —CH(CH₃)—SO₂—CH₃, —CH(CH₂CH₃)—SO₂—CH₃, —CH((CH₂)₂CH₃)—SO₂—CH₃, —CH(CH(CH₃)₂)—SO₂—CH₃, —CH(CN)—SO₂—CH₃, —CH(CHO)—SO₂—CH₃, —CH(NH₂)—SO₂—CH₃, —CH(NO₂)—SO₂—CH₃, —CH(F)—SO₂—CH₃, —CH(Cl)—SO₂—CH₃, —CH(Br)—SO₂—CH₃, —CH(I)—SO₂—CH₃, —CH₂—SO₂—CH₂—CH₃, —CH(CH₃)—SO₂—CH₂—CH₃, —CH(CH₂CH₃)—SO₂—CH₂—CH₃, —CH((CH₂)₂CH₃)—SO₂—CH₂—CH₃, —CH(CH(CH₃)₂)—SO₂—CH₂—CH₃, —CH(CN)—SO₂—CH₂—CH₃, —CH(CHO)—SO₂—CH₂—CH₃, —CH(NH₂)—SO₂—CH₂—CH₃, —CH(NO₂)—SO₂—CH₂—CH₃, —CH(F)—SO₂—CH₂—CH₃, —CH(Cl)—SO₂—CH₂—CH₃, —CH(Br)—SO₂—CH₂—CH₃, —CH(I)—SO₂—CH₂—CH₃, —CH₂—SO₂—CH(CHO)—CH₃, —CH(CH₃)—SO₂—CH(CHO)—CH₃, —CH(CH₂CH₃)—SO₂—CH(CHO)—CH₃, —CH((CH₂)₂CH₃)—SO₂—CH(CHO)—CH₃, —CH(CH(CH₃)₂)—SO₂—CH(CHO)—CH₃, —CH(CN)—SO₂—CH(CHO)—CH₃, —CH(CHO)—SO₂—CH(CHO)—CH₃, —CH(NH₂)—SO₂—CH(CHO)—CH₃, —CH(NO₂)—SO₂—CH(CHO)—CH₃, —CH(F)—SO₂—CH(CHO)—CH₃, —CH(Cl)—SO₂—CH(CHO)—CH₃, —CH(Br)—SO₂—CH(CHO)—CH₃, —CH(I)—SO₂—CH(CHO)—CH₃, —CH₂—SO₂—CH₂CH₃, —CH(CH₃)—SO₂—CH₂CH₃, —CH(CH₂CH₃)—SO₂—CH₂CH₃, —CH((CH₂)₂CH₃)—SO₂—CH₂CH₃, —CH(CH(CH₃)₂)—SO₂—CH₂CH₃, —CH(CN)—SO₂—CH₂CH₃, —CH(CHO)—SO₂—CH₂CH₃, —CH(NH₂)—SO₂—CH₂CH₃, —CH(NO₂)—SO₂—CH₂CH₃, —CH(F)—SO₂—CH₂CH₃, —CH(Cl)—SO₂—CH₂CH₃, —CH(Br)—SO₂—CH₂CH₃, —CH(I)—SO₂—CH₂CH₃, —CH₂—SO₂—CH₂—CH₂CH₃, —CH(CH₃)—SO₂—CH₂—CH₂CH₃, —CH(CH₂CH₃)—SO₂—CH₂—CH₂CH₃, —CH((CH₂)₂CH₃)—SO₂—CH₂—CH₂CH₃, —CH(CH(CH₃)₂)—SO₂—CH₂—CH₂CH₃, —CH(CN)—SO₂—CH₂—CH₂CH₃, —CH(CHO)—SO₂—CH₂—CH₂CH₃, —CH(NH₂)—SO₂—CH₂—CH₂CH₃, —CH(NO₂)—SO₂—CH₂—CH₂CH₃, —CH(F)—SO₂—CH₂—CH₂CH₃, —CH (Cl)—SO₂—CH₂—CH₂CH₃, —CH(Br)—SO₂—CH₂—CH₂CH₃, —CH(I)—SO₂—CH₂—CH₂CH₃, —CH₂—SO₂—CH(CHO)—CH₂CH₃, —CH(CH₃)—SO₂—CH(CHO)—CH₂CH₃, —CH(CH₂CH₃)—SO₂—CH (CHO)—CH₂CH₃, —CH((CH₂)₂CH₃)—SO₂—CH(CHO)—CH₂CH₃, —CH(CH(CH₃)₂)—SO₂—CH(CHO)—CH₂CH₃, —CH(CN)—SO₂—CH(CHO)—CH₂CH₃, —CH(CHO)—SO₂—CH(CHO)—CH₂CH₃, —CH(NH₂)—SO₂—CH(CHO)—CH₂CH₃, —CH(NO₂)≧SO₂—CH(CHO)—CH₂CH₃, —CH(F)—SO₂—CH(CHO)—CH₂CH₃, —CH(Cl)—SO₂—CH(CHO)—CH₂CH₃, —CH(Br)—SO₂—CH(CHO)—CH₂CH₃, —CH(I)—SO₂—CH(CHO)—CH₂CH₃, —CH₂—SO₂—(CH₂)₂CH₃, —CH(CH₃)—SO₂—(CH₂)₂CH₃, —CH(CH₂CH₃)—SO₂—(CH₂)₂CH₃, —CH((CH₂)₂CH₃)—SO₂—(CH₂)₂CH₃, —CH(CH(CH₃)₂)—SO₂—(CH₂)₂CH₃, —CH(CN)—SO₂—(CH₂)₂CH₃, —CH(CHO)—SO₂—(CH₂)₂CH₃, —CH(NH₂)—SO₂—(CH₂)₂CH₃, —CH(NO₂)—SO₂—(CH₂)₂CH₃, —CH(F)—SO₂—(CH₂)₂CH₃, —CH(Cl)—SO₂—(CH₂)₂CH₃, —CH(Br)—SO₂—(CH₂)₂CH₃, —CH(I)—SO₂—(CH₂)₂CH₃, —CH₂—SO₂—CH₂—(CH₂)₂CH₃, —CH(CH₃)—SO₂—CH₂—(CH₂)₂CH₃, —CH(CH₂CH₃)—SO₂—CH₂—(CH₂)₂CH₃, —CH((CH₂)₂CH₃)—SO₂—CH₂—(CH₂)₂CH₃, —CH(CH(CH₃)₂)—SO₂—CH₂—(CH₂)₂CH₃, —CH(CN)—SO₂—CH₂—(CH₂)₂CH₃, —CH(CHO)—SO₂—CH₂—(CH₂)₂CH₃, —CH(NH₂)—SO₂—CH₂—(CH₂)₂CH₃, —CH(NO₂)—SO₂—CH₂—(CH₂)₂CH₃, —CH(F)—SO₂—CH₂—(CH₂)₂CH₃, —CH(Cl)—SO₂—CH₂—(CH₂)₂CH₃, —CH(Br)—SO₂—CH₂—(CH₂)₂CH₃, —CH(I)—SO₂—CH₂—(CH₂)₂CH₃, —CH₂—SO₂—CH(CHO)—(CH₂)₂CH₃, —CH(CH₃)—SO₂—CH(CHO)—(CH₂)₂CH₃, —CH(CH₂CH₃)—SO₂—CH(CHO)—(CH₂)₂CH₃, —CH((CH₂)₂CH₃)—SO₂—CH(CHO)—(CH₂)₂CH₃, —CH(CH(CH₃)₂)—SO₂—CH(CHO)—(CH₂)₂CH₃, —CH(CN)—SO₂—CH(CHO)—(CH₂)₂CH₃, —CH(CHO)—SO₂—CH(CHO)—(CH₂)₂CH₃, —CH(NH₂)—SO₂—CH(CHO)—(CH₂)₂CH₃, —CH(NO₂)—SO₂—CH(CHO)—(CH₂)₂CH₃, —CH(F)—SO₂—CH(CHO)—(CH₂)₂CH₃, —CH(Cl)—SO₂—CH(CHO)—(CH₂)₂CH₃, —CH(Br)—SO₂—CH(CHO)—(CH₂)₂CH₃, —CH(I)—SO₂—CH(CHO)—(CH₂)₂CH₃, —CH₂—SO₂—CH(CH₃)₂, —CH(CH₃)—SO₂—CH(CH₃)₂, —CH(CH₂CH₃)—SO₂—CH(CH₃)₂, —CH((CH₂)₂CH₃)—SO₂—CH(CH₃)₂, —CH(CH(CH₃)₂)—SO₂—CH(CH₃)₂, —CH(CN)—SO₂—CH(CH₃)₂, —CH(CHO)—SO₂—CH(CH₃)₂, —CH(NH₂)—SO₂—CH(CH₃)₂, —CH(NO₂)—SO₂—CH(CH₃)₂, —CH(F)—SO₂—CH(CH₃)₂, —CH(Cl)—SO₂—CH(CH₃)₂, —CH(Br)—SO₂—CH(CH₃)₂, —CH(I)—SO₂—CH(CH₃)₂, —CH₂—SO₂—CH₂—CH(CH₃)₂, —CH(CH₃)—SO₂—CH₂—CH(CH₃)₂, —CH(CH₂CH₃)—SO₂—CH₂—CH(CH₃)₂, —CH((CH₂)₂CH₃)—SO₂—CH₂—CH(CH₃)₂, —CH(CH(CH₃)₂)—SO₂—CH₂—CH(CH₃)₂, —CH(CN)—SO₂—CH₂—CH(CH₃)₂, —CH(CHO)—SO₂—CH₂—CH(CH₃)₂, —CH(NH₂)—SO₂—CH₂—CH(CH₃)₂, —CH(NO₂)—SO₂—CH₂—CH(CH₃)₂, —CH(F)—SO₂—CH₂—CH(CH₃)₂, —CH(Cl)—SO₂—CH₂—CH (CH₃)₂, —CH(Br)—SO₂—CH₂—CH(CH₃)₂, —CH(I)—SO₂—CH₂—CH(CH₃)₂, —CH₂—SO₂—CH(CHO)—CH(CH₃)₂, —CH(CH₃)—SO₂—CH(CHO)—CH(CH₃)₂, —CH(CH₂CH₃)—SO₂—CH(CHO)—CH(CH₃)₂, —CH((CH₂)₂CH₃)—SO₂—CH(CHO)—CH(CH₃)₂, —CH(CH(CH₃)₂)—SO₂—CH(CHO)—CH(CH₃)₂, —CH(CN)—SO₂—CH(CHO)—CH(CH₃)₂, —CH(CHO)—SO₂—CH(CHO)—CH(CH₃)₂, —CH(NH₂)—SO₂—CH(CHO)—CH(CH₃)₂, —CH(NO₂)—SO₂—CH(CHO)—CH(CH₃)₂, —CH(F)—SO₂—CH(CHO)—CH(CH₃)₂, —CH(Cl)—SO₂—CH(CHO)—CH(CH₃)₂, —CH(Br)—SO₂—CH(CHO)—CH(CH₃)₂, —CH(I)—SO₂—CH(CHO)—CH(CH₃)₂, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃, —CH₂NH₂, CH₂NO₂, CH₂F, CH₂Cl, CH₂Br, CH₂I, CHF₂, CHCl₂, CHBr₂, CHI₂, CF₃, CCl₃, CBr₃, Cl₃, -phenyl.

Particularly preferred agents according to the invention for dyeing and/or lightening keratin fibers are characterized in that they contain one or more aldehydes from the group acetaldehyde, trichloroacetaldehyde, trifluoroacetaldehyde, nitroacetaldehyde, methylsulfonylacetaldehyde or benzaldehyde.

The aldehyde(s) are preferably used within narrow quantity ranges. Preferred agents according to the invention for dyeing and/or lightening keratin fibers are here those which contain the aldehyde(s) in quantities of 0.1 to 12.5 wt. %, preferably of 0.25 to 10 wt. %, particularly preferably of 0.5 to 7.5 wt. % and in particular from 1 to 5 wt. %, in each case relative to the entire agent.

The agents according to the invention particularly preferentially additionally contain hydrogen peroxide. Particularly preferred agents according to the invention for dyeing and/or lightening keratin fibers are here those which contain 0.5 to 15 wt. %, preferably 1 to 12.5 wt. %, particularly preferably 2.5 to 10 wt % and in particular 3 to 6 wt. % of hydrogen peroxide (calculated as 100% H₂O₂)

The hydrogen peroxide may also be used in the form of the addition compounds thereof onto solid carriers; hydrogen peroxide itself is preferably used. The hydrogen peroxide is used as a solution or in the form of a solid addition compound of hydrogen peroxide onto inorganic or organic compounds, such as for example sodium perborate, sodium percarbonate, magnesium percarbonate, sodium percarbamide, polyvinylpyrrolidone n H₂O₂ (n is a positive integer greater than 0), urea peroxide and melamine peroxide.

Aqueous hydrogen peroxide solutions are very particularly preferred according to the invention. The concentration of a hydrogen peroxide solution is determined on the one hand by statutory requirements and on the other hand by the desired effect; 6 to 12 percent solutions in water are preferably used.

The agents according to the invention may additionally contain further oxidizing agents, which are also known as “boosters”. Selection of these further peroxo compounds is not in principle subject to any restrictions; conventional peroxo compounds known to a person skilled in the art are for example ammonium peroxydisulfate, potassium peroxydisulfate, sodium peroxydisulfate, ammonium persulfate, potassium persulfate, sodium persulfate, percarbonates such as magnesium percarbonate, peroxides such as barium peroxide as well as perborates, urea peroxide and melamine peroxide. Among these peroxo compounds, which may also be used in combination, it is the inorganic compounds which are preferred according to the invention. Peroxydisulfates, in particular combinations of at least two peroxydisulfates, are particularly preferred. Preferred agents according to the invention for dyeing and/or lightening keratin fibers are here those which additionally contain 0.01 to 2 wt. % of at least one solid peroxo compound, which is selected from ammonium, alkali metal and alkaline earth metal persulfates, peroxomonosulfates and peroxydisulfates, preferred agents containing peroxydisulfates which are preferably selected from sodium peroxydisulfate and/or potassium peroxydisulfate and/or ammonium peroxydisulfate and particularly preferred agents containing at least two different peroxydisulfates.

Persulfates are furthermore particularly preferred, in particular the mixture of potassium peroxosulfate, potassium hydrogensulfate and potassium sulfate known as Caro's salt.

The agents according to the invention serve as agents for dyeing and/or lightening keratin fibers. Pure lightening agents, which are also known as blonding agents, preferably additionally contain oxidizing agents. The agents according to the invention are preferably provided as dyes which not (only) serve for lightening, but also or exclusively for modifying the color of the keratin fibers treated therewith. Agents which have a simultaneous coloring and lightening action are also described as lightening dyes.

The agents according to the invention are preferably dyes, i.e. agents for modifying the color of keratin fibers. The agents according to the invention here contain no “oxidation dye precursors”, i.e. dye precursors of the coupler and developer type, such that dyeing is achieved by the use of direct dyes. Agents for dyeing and/or lightening keratin fibers which are preferred according to the invention are thus characterized in that they additionally contain, relative to the weight thereof, 0.001 to 5 wt. % of one or more direct dyes.

Direct dyes are conventionally nitrophenylenediamines, nitroaminophenols, azo dyes, anthraquinones or indophenols, triphenylmethane dyes, acidic dyes, basic dyes, preferably from the group of compounds known by the international names or trade names HC Yellow 2, HC Yellow 4, HC Yellow 5, HC Yellow 6, HC Yellow 12, HC Yellow 13, Acid Yellow 1, Acid Yellow 10, Acid Yellow 23, Acid Yellow 36, HC Orange 1, Disperse Orange 3, Acid Orange 7, HC Red 1, HC Red 3, HC Red 10, NC Red 11, HC Red 13, Acid Red 33, Acid Red 52, Acid Red 92, HC Red BN, Pigment Red 57:1, HC Blue 2, NC Blue 12, Disperse Blue 3, Disperse Red 17, Acid Blue 7, Acid Green 50, HC Violet 1, Disperse Violet 1, Disperse Violet 4, Acid Violet 43, Disperse Black 9, Acid Black 1, and Acid Black 52 as well as 1,4-diamino-2-nitrobenzene, 2-amino-4-nitrophenol, 1,4-bis-(f3-hydroxyethyl)amino-2-nitro-benzene, 3-nitro-4-(β-hydroxyethypaminophenol, 2-(2-hydroxyethyl)amino-4,6-dinitrophenol, 1-(2′-hydroxyethyl)amino-4-methyl-2-nitrobenzene, 1-amino-4-(2-hydroxyethyl)amino-5-chloro-2-nitrobenzene, 4-amino-3-nitrophenol, 1-(2-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid and 2-chloro-6-ethylamino-1-hydroxy-4-nitrobenzene, 3-(2′,6′-diaminopyridyl-3′-azo)-pyridine; 2-((4-(ethyl(2-hydroxyethyl)-amino)-2-methylphenyl)azo)-5-nitro-1,3-thiazole; N′,N-di-(2-hydroxyethyl)-3-methyl-4-((4-nitrophenol)azo)-aniline; 3-diethylamino-7-(4-dimethylaminophenylazo)-5-phenylphenazinium chloride; 4-(2-thiazolylazo)-resorcinol; 4-(((4-phenylamino)azo)benzenesulfonic acid sodium salt; 1-((3-aminopropyl)amino)-9,10-anthracenedione; 3′,3″,4,5,5′,5″,6,7-octabromo-phenolsulfonaphthalein; 1-((4-amino-3,5-dimethylphenyl)-(2,6-dichlorophenyl)-methylene)-3,5-dimethyl-4-imino-2,5-cyclohexadiene phosphoric acid (1:1) (Basic Blue 77); 3′,3″,5′,5″-tetrabromo-m-cresolsulfonaphthalein; 2,4-dinitro-1-naphthol-7-sulfonic acid disodium salt; 4-[2′-hydroxy-1′-naphthyl)azo]-benzenesulfonic acid sodium salt; 3′,6′-dihydroxy-2′,4′,5′,7′-tetraiodospiro-[iso-benzofuran-1(3H), g1(9H)-xanthen]-3-one disodium salt; 6-hydroxy-5-((2-methoxy-5-methyl-4-sulfophenyl)azo)-2-naphthalenesulfonic acid disodium salt; 2,4-dinitro-1-naphthol sodium salt; 2′,4′,5′,7′-tetrabromo-4,5,6,7-tetrachloro-3′,6′-dihydroxy-spiro[isobenzofuran-1(3H),9′[9H]xanthen]-3-one disodium salt; 4-(2-hydroxy-1-naphthylazo)-3-methylbenzenesulfonic acid sodium salt; Basic Orange 31, Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Red 2, Basic Red 12 Basic Red 22, Basic Red 51, Basic Red 76, Basic Violet 1, Basic Violet 2, Basic Violet 3, Basic Violet 10, Basic Violet 14, Basic Yellow 57, Basic Yellow 87, Basic Red 46.

Agents for dyeing and/or lightening keratin fibers which are preferred according to the invention are characterized in that they contain at least one direct dye which is selected from nitrophenylenediamines, nitroaminophenols, azo dyes, anthraquinones or indophenols, preferably from the group of dyes known by the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, Acid Black 52, Basic Yellow 87, Basic Orange 31 and Basic Red 51 as well as 1,4-diamino-2-nitrobenzene, 2-amino-4-nitrophenol, 1,4-bis-(β-hydroxy-ethyl)amino-2-nitrobenzene, 3-nitro-4-(β-hydroxyethyl)aminophenol, 2-(2-hydroxyethyl)amino-4,6-dinitrophenol, 1-(2′-hydroxyethyl)amino-4-methyl-2-nitrobenzene, 1-amino-4-(2-hydroxyethyl)amino-5-chloro-2-nitrobenzene, 4-amino-3-nitrophenol, 1-(2-ureidoethyl)amino-4-nitrobenzene, 4-amino-2-nitro-diphenylamine-2′-carboxylic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid and 2-chloro-6-ethylamino-1-hydroxy-4-nitrobenzene.

The agents according to the invention may furthermore contain a cationic direct dye. Particular preference is here given to
(a) cationic triphenylmethane dyes, such as for example Basic Blue 7, Basic Blue 26, Basic Violet 2 and Basic Violet 14,
(b) aromatic systems substituted with a quaternary nitrogen group, such as for example Basic Yellow 57, Basic Red 76, Basic Blue 99, Basic Brown 16 and Basic Brown 17, as well as
(c) direct dyes containing a heterocycle which comprises at least one quaternary nitrogen atom, as are for example mentioned in claims 6 to 11 in EP-A2-998 908, to which explicit reference is here made.

Preferred cationic direct dyes of group (c) are in particular the following compounds:

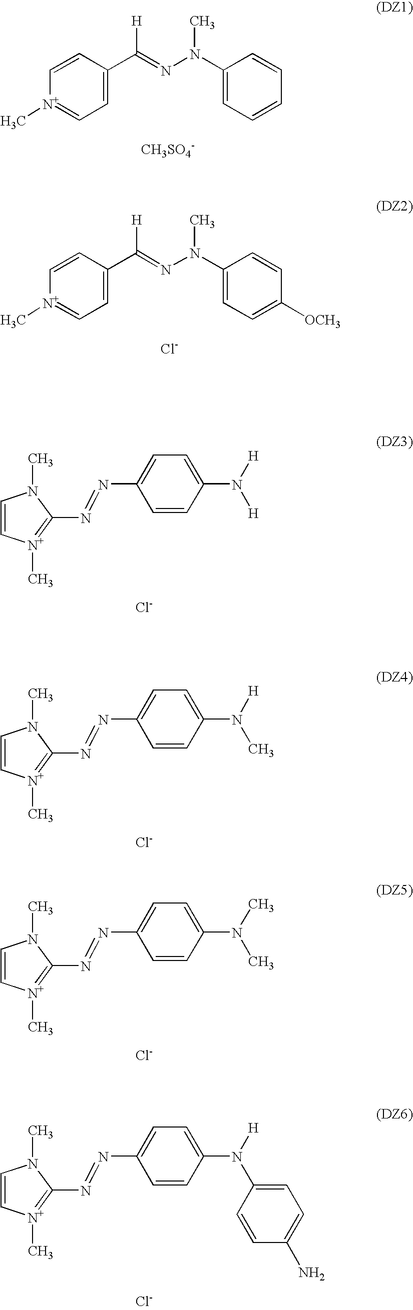

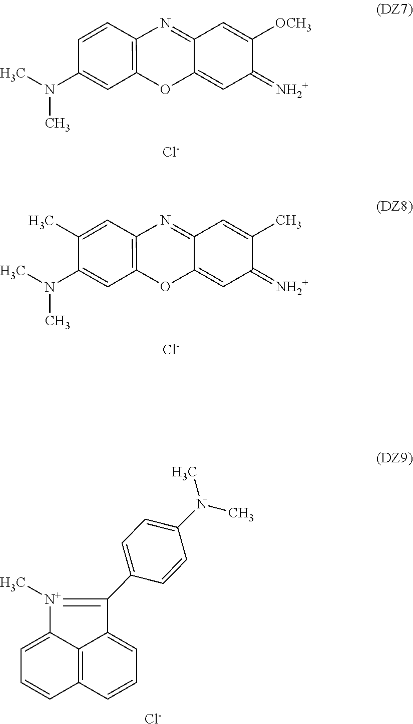

The compounds of the formulae (DZ1), (DZ3) and (DZ5), which are also known by the names Basic Yellow 87, Basic Orange 31 and Basic Red 51, are very particularly preferred cationic direct dyes of group (c).

The cationic direct dyes distributed under the trademark Arianor® are cationic direct dyes which are likewise very particularly preferred according to the invention.

The agents according to the invention according to this embodiment preferably contain the direct dyes in a quantity of 0.01 to 20 wt. %, relative to the entire dye.

Furthermore, the preparations according to the invention may also contain naturally occurring dyes such as are contained for example in henna red, henna neutral, henna black, chamomile flowers, sandalwood, black tea, alder buckthorn bark, sage, logwood, madder root, catechu, lotus tree and alkanet root.

It is not necessary for the direct dyes in each case to be uniform compounds. Instead, as a result of the production processes for the individual dyes, the hair colorants may contain subordinate quantities of still further components, provided that these do not have a disadvantageous effect on the dyeing result or must be excluded for other, for example toxicological, reasons.

Keratin fibers may also be dyed by means of dyes which are formed in an oxidatively catalysed reaction of CH-acidic compounds with reactive carbonyl compounds.

In a further embodiment the agent according to the invention therefore contains a combination of component
A compounds which contain a reactive carbonyl group with component
B compounds selected from (a) CH-acidic compounds, (b) compounds with a primary or secondary amino group or hydroxy group, selected from primary or secondary aromatic amines, nitrogenous heterocyclic compounds and aromatic hydroxy compounds, (c) amino acids, (d) oligopeptides made up of 2 to 9 amino acids.

Compounds according to the invention with a reactive carbonyl group (hereinafter also referred to as reactive carbonyl compounds or component A) have at least one carbonyl group as a reactive group which reacts with the component B compounds to form a chemical bond which links the two components together. According to the invention, component A also includes those compounds in which the reactive carbonyl group is derivatized or masked in such a manner that the reactivity of the carbon atom of the derivatized or masked carbonyl group towards component B is always present. These derivatives are preferably condensation compounds of reactive carbonyl compounds with
a) amines and the derivatives thereof resulting in the formation of imines or oximes as the condensation compound
b) alcohols resulting in the formation of acetals or ketals as the condensation compound
c) water resulting in the formation of hydrates as an addition compound.

Component A is preferably selected from the group composed of acetophenone, propiophenone, 2-hydroxyacetophenone, 3-hydroxyacetophenone, 4-hydroxyacetophenone, 2-hydroxypropiophenone, 3-hydroxypropiophenone, 4-hydroxypropiophenone, 2-hydroxybutyrophenone, 3-hydroxybutyrophenone, 4-hydroxybutyrophenone, 2,4-dihydroxyacetophenone, 2,5-dihydroxyacetophenone, 2,6-dihydroxyacetophenone, 2,3,4-trihydroxyacetophenone, 3,4,5-trihydroxyacetophenone, 2,4,6-trihydroxyacetophenone, 2,4,6-trimethoxyacetophenone, 3,4,5-trimethoxyacetophenone, 3,4,5-trimethoxyacetophenone diethyl ketal, 4-hydroxy-3-methoxyacetophenone, 3,5-dimethoxy-4-hydroxyacetophenone, 4-aminoacetophenone, 4-dimethylaminoacetophenone, 4-morpholinoacetophenone, 4-piperidinoacetophenone, 4-imidazolinoacetophenone, 2-hydroxy-5-bromoacetophenone, 4-hydroxy-3-nitroacetophenone, acetophenone-2-carboxylic acid, acetophenone-4-carboxylic acid, benzophenone, 4-hydroxybenzophenone, 2-aminobenzophenone, 4,4′-dihydroxybenzophenone, 2,4-dihydroxybenzophenone, 2,4,4-trihydroxybenzophenone, 2,3,4-trihydroxybenzophenone, 2-hydroxy-1-acetonaphthone, 1-hydroxy-2-acetonaphthone, chromone, chromone-2-carboxylic acid, flavone, 3-hydroxyflavone, 3,5,7-trihydroxyflavone, 4′,5,7-trihydroxyfravone, 5,6,7-trihydroxyflavone, quercetin, 1-indanone, 9-fluorenone, 3-hydroxyfluorenone, anthrone, 1,8-dihydroxyanthrone, vanillin, coniferyl aldehyde, 2-methoxybenzaldehyde, 3-methoxybenzaldehyde, 4-methoxybenzaldehyde, 2-ethoxybenzaldehyde, 3-ethoxybenzaldehyde, 4-ethoxybenzaldehyde, 4-hydroxy-2,3-dimethoxybenzaldehyde, 4-hydroxy-2,5-dimethoxybenzaldehyde, 4-hydroxy-2,6-dimethoxybenzaldehyde, 4-hydroxy-2-methylbenzaldehyde, 4-hydroxy-3-methylbenzaldehyde, 4-hydroxy-2,3-dimethylbenzaldehyde, 4-hydroxy-2,5-dimethylbenzaldehyde, 4-hydroxy-2,6-dimethylbenzaldehyde, 4-hydroxy-3,5-dimethoxybenzaldehyde, 4-hydroxy-3,5-dimethylbenzaldehyde, 3,5-diethoxy-4-hydroxybenzaldehyde, 2,6-diethoxy-4-hydroxybenzaldehyde, 3-hydroxy-4-methoxybenzaldehyde, 2-hydroxy-4-methoxybenzaldehyde, 2-ethoxy-4-hydroxybenzaldehyde, 3-ethoxy-4-hydroxybenzaldehyde, 4-ethoxy-2-hydroxybenzaldehyde, 4-ethoxy-3-hydroxybenzaldehyde, 2,3-dimethoxybenzaldehyde, 2,4-dimethoxybenzaldehyde, 2,5-dimethoxybenzaldehyde, 2,6-dimethoxybenzaldehyde, 3,4-dimethoxybenzaldehyde, 3,5-dimethoxybenzaldehyde, 2,3,4-trimethoxybenzaldehyde, 2,3,5-trimethoxybenzaldehyde, 2,3,6-trimethoxybenzaldehyde, 2,4,6-trimethoxybenzaldehyde, 2,4,5-trimethoxybenzaldehyde, 2,5,6-trimethoxybenzaldehyde, 2-hydroxybenzaldehyde, 3-hydroxybenzaldehyde, 4-hydroxybenzaldehyde, 2,3-dihydroxybenzaldehyde, 2,4-dihydroxybenzaldehyde, 2,5-dihydroxybenzaldehyde, 2,6-dihydroxybenzaldehyde, 3,4-dihydroxybenzaldehyde, 3,5-dihydroxybenzaldehyde, 2,3,4-trihydroxybenzaldehyde, 2,3,5-trihydroxybenzaldehyde, 2,3,6-trihydroxybenzaldehyde, 2,4,6-trihydroxybenzaldehyde, 2,4,5-trihydroxybenzaldehyde, 2, 5,6-trihydroxybenzaldehyde, 4-hydroxy-2-methoxybenzaldehyde, 4-dimethylaminobenzaldehyde, 4-diethylaminobenzaldehyde, 4-dimethylamino-2-hydroxybenzaldehyde, 4-diethylamino-2-hydroxybenzaldehyde, 4-pyrrolidinobenzaldehyde, 4-morpholinobenzaldehyde, 2-morpholinobenzaldehyde, 4-piperidinobenzaldehyde, 2-methoxy-1-naphthaldehyde, 4-methoxy-1-naphthaldehyde, 2-hydroxy-1-naphthaldehyde, 2,4-dihydroxy-1-napthaldehyde, 4-hydroxy-3-methoxy-1-naphthaldehyde, 2-hydroxy-4-methoxy-1-naphthaldehyde, 3-hydroxy-4-methoxy-1-naphthaldehyde, 2,4-dimethoxy-1-naphthaldehyde, 3,4-dimethoxy-1-naphthaldehyde, 4-hydroxy-1-naphthaldehyde, 4-dimethylamino-1-naphthaldehyde, 2-methoxycinnamaldehyde, 4-methoxycinnamaldehyde, 4-hydroxy-3-methoxycinnamaldehyde, 3,5-dimethoxy-4-hydroxycinnamaldehyde, 4-dimethylaminocinnamaldehyde, 2-dimethylaminobenzaldehyde, 2-chloro-4-dimethylaminobenzaldehyde, 4-dimethylamino-2-methylbenzaldehyde, 4-diethylaminocinnamaldehyde, 4-dibutylaminobenzaldehyde, 4-diphenylaminobenzardehyde, 4-dimethylamino-2-methoxybenzaldehyde, 4-(1-imidazolyl)-benzaldehyde, piperonal, 2,3,6,7-tetrahydro-1H,5H-benzo[ij]quinolizine-9-carboxaldehyde, 2,3,6,7-tetrahydro-8-hydroxy-1H , 5H-benzo[ij]quinolizine-9-carboxaldehyde, N-ethylcarbazole-3-aldehyde, 2-formylmethylene-1,3,3-trimethylindoline (Fischer's aldehyde or tribasic aldehyde), 2-indolealdehyde, 3-indolealdehyde, 1-methylindole-3-aldehyde, 2-methylindole-3-aldehyde, 1-acetylindole-3-aldehyde, 3-acetylindole, 1-methyl-3-acetylindole, 2-(1′,3′,3′-trimethyl-2-indolinylidene)-acetaldehyde, 1-methylpyrrole-2-aldehyde, 1-methyl-2-acetylpyrrole, 4-pyridinealdehyde, 2-pyridinealdehyde, 3-pyridinealdehyde, 4-acetylpyridine, 2-acetylpyridine, 3-acetylpyridine, pyridoxal, quinoline-3-aldehyde, quinoline-4-aldehyde, antipyrine-4-aldehyde, furfural, 5-nitrofurfural, 2-thenoyltrifluoroacetone, chromone-3-aldehyde, 3-(5′-nitro-2′-furyl)-acrolein, 3-(2′-furyl)-acrolein and imidazole-2-aldehyde, 1,3-diacetylbenzene, 1,4-diacetylbenzene, 1,3,5-triacetylbenzene, 2-benzoylacetophenone, 2-(4′-methoxybenzoyl)-acetophenone, 2-(2′-furoyl)-acetophenone, 2-(2′-pyridoyl)-acetophenone and 2-(3′-pyridoyl)-acetophenone, benzylideneacetone, 4-hydroxybenzylideneacetone, 2-hydroxybenzylideneacetone, 4-methoxybenzylideneacetone, 4-hydroxy-3-methoxybenzylideneacetone, 4-dimethylaminobenzylideneacetone, 3,4-methylenedioxybenzylideneacetone, 4-pyrrolidinobenzylideneacetone, 4-piperidinobenzylideneacetone, 4-morphollnobenzylideneacetone, 4-diethylaminobenzylideneacetone, 3-benzylidene-2,4-pentanedione, 3-(4′-hydroxybenzylidene)-2,4-pentanedione, 3-(4′-dimethylaminobenzylidene)-2,4-pentanedione, 2-benzylidenecyclohexanone, 2-(4′-hydroxybenzylidene)-cyclohexanone, 2-(4′-dimethylaminobenzylidene)-cyclohexanone, 2-benzylidene-1,3-cyclohexanedione, 2-(4′-hydroxybenzylidene)-1,3-cyclohexanedione, 3-(4′-dimethylaminobenzylidene)-1,3-cyclohexanedione, 2-benzylidene-5,5-dimethyl-1,3-cyclohexanedione, 2-(4′-hydroxybenzylidene)-5,5-dimethyl-1,3-cyclohexanedione, 2-(4′-hydroxy-3-methoxybenzylidene)-5,5-dimethyl-1,3-cyclohexanedione, 2-(4′-dimethylaminobenzylidene)-5,5-dimethyl-1,3-cyclohexanedlone, 2-benzylidenecyclopentanone, 2′-(4-hydroxybenzylidene)-cyclopentanone, 2-(4′-dimethylaminobenzylidene)-cyclopentanone, 5-(4-dimethylaminophenyl)-penta-2,4-dienal, 5-(4-diethylaminophenyl)penta-2,4-dienal, 5-(4-methoxyphenyl)penta-2,4-dienal, 5-(3,4-dimethoxyphenyl)penta-2,4-dienal, 5-(2,4-dimethoxyphenyl)penta-2,4-dienal, 5-(4-piperidinophenyl)penta-2,4-dienal, 5-(4-morpholinophenyl)penta-2,4-dienal, 5-(4-pyrrolidinophenyl)penta-2,4-dienal, 6-(4-dimethylaminophenyl)hexa-3,5-dien-2-one, 6-(4-diethylaminophenyl)hexa-3,5-dien-2-one, 6-(4-methoxyphenyl)hexa-3,5-dien-2-one, 6-(3,4-dimethoxyphenyl)hexa-3,5-dien-2-one, 6-(2,4-dimethoxyphenyl)hexa-3,5-dien-2-one, 6-(4-piperidinophenyl)hexa-3,5-dien-2-one, 6-(4-morpholinophenyl)hexa-3,5-dien-2-one, 6-(4-pyrrolidinophenyl)hexa-3,5-dien-2-one, 5-(4-dimethylamino-1-naphthyl)penta-3,5-dienal, 2-nitrobenzaldehyde, 3-nitrobenzaldehyde, 4-nitrobenzaldehyde, 4-methyl-3-nitrobenzaldehyde, 3-hydroxy-4-nitrobenzaldehyde, 4-hydroxy-3-nitrobenzaldehyde, 5-hydroxy-2-nitrobenzaldehyde, 2-hydroxy-5-nitrobenzaldehyde, 2-hydroxy-3-nitrobenzaldehyde, 2-fluoro-3-nitrobenzaldehyde, 3-methoxy-2-nitrobenzaldehyde, 4-chloro-3-nitrobenzaldehyde, 2-chloro-6-nitrobenzaldehyde, 5-chloro-2-nitrobenzaldehyde, 4-chloro-2-nitrobenzaldehyde, 2,4-dinitrobenzaldehyde, 2,6-dinitrobenzaldehyde, 2-hydroxy-3-methoxy-5-nitrobenzaldehyde, 4,5-dimethoxy-2-nitrobenzaldehyde, 6-nitropiperonal, 2-nitropiperonal, 5-nitrovanillin, 2,5-dinitrosalicylaldehyde, 5-bromo-3-nitrosalicylaldehyde, 3-nitro-4-formyl-benzenesulfonic acid, 4-nitro-1-naphthaidehyde, 2-nitrocinnamaldehyde, 3-nitrocinnamaldehyde, 4-nitrocinnamaldehyde, 9-methyl-3-carbazolealdehyde, 9-ethyl-3-carbazolealdehyde, 3-acetylcarbazole, 3,6-diacetyl-9-ethylcarbazole, 3-acetyl-9-methylcarbazole, 1,4-dimethyl-3-carbazolealdehyde, 1,4,9-trimethyl-3-carbazolealdehyde, 4-formyl-1-methylpyridinium, 2-formyl-1-methylpyridinium, 4-formyl-1-ethylpyridinium, 2-formyl-1-ethylpyridinium, 4-formyl-1-benzylpyridinium, 2-formyl-1-benzylpyridinium, 4-formyl-1,2-dimethylpyridinium, 4-formyl-1,3-dimethylpyridinium, 4-formyl-1-methylquinolinium, 2-formyl-1-methylquinolinium, 4-acetyl-1-methylpyridinium, 2-acetyl-1-methylpyridinium, 4-acetyl-1-methylquinolinium, 5-formyl-1-methylquinolinium, 6-formyl-1-methylquinolinium, 7-formyl-1-methylquinolinium, 8-formyl-1-methylquinolinium, 5-formyl-1-ethylquinolinium, 6-formyl-1-ethylquinolinium, 7-formyl-1-ethylquinolinium, 8-formyl-1-ethylquinolinium, 5-formyl-1-benzylquinolinium, 6-formyl-1-benzylquinolinium, 7-formyl-1-benzylquinolinium, 8-formyl-1-benzylquinoiinium, 5-formyl-1-allylquinolinium, 6-formyl-1-allylquinolinium, 7-formyl-1-allylquinolinium and 8-formyl-1-allylquinolinium, 5-acetyl-1-methylquinolinium, 6-acetyl-1-methylquinolinium, 7-acetyl-1-methylquinolinium, 8-acetyl-1-methylquinolinium, 5-acetyl-1-ethylquinolinium, 6-acetyl-1-ethylquinolinium, 7-acetyl-1-ethylquinolinium, 8-acetyl-1-ethylquinolinium, 5-acetyl-1-benzylquinolinium, 6-acetyl-1-benzylquinolinium, 7-acetyl-1-benzylquinolinium, 8-acetyl-1-benzylquinolinium, 5-acetyl-1-allylquinolinium, 6-acetyl-1-allyiquinolinium, 7-acetyl-1-allylquinolinium and 8-acetyl-1-allylquinolinium, 9-formyl-10-methylacridinium, 4-(2′-formylvinyl)-1-methylpyridinium, 1,3-dimethyl-2-(4′-formylphenyl)-benzimidazolium, 1,3-dimethyl-2-(4′-formylphenyl)-imidazolinium, 2-(4′-formylphenyl)-3-methylbenzothiazolium, 2-(4′-acetylphenyl)-3-methylbenzothiazolium, 2-(4′-formylphenyl)-3-methylbenzoxazolium, 2-(5′-formyl-2′-furyl)-3-methylbenzothiazolium, 2-(5′-formyl-2′-furyl)-3-methylbenzothiazolium, 2-(5t-formyl-2′-thienyl)-3-methylbenzothiazolium, 2-(3′-formylphenyl)-3-methylbenzothiazolium, 2-(4′-formyl-1-naphthyl)-3-methylbenzothiazolium, 5-chloro-2-(4′-formylphenyl)-3-methylbenzothiazolium, 2-(4′-formylphenyl)-3,5-dimethylbenzothiazolium benzenesulfonate, p-toluenesulfonate, methanesulfonate, perchlorate, sulfate, chloride, bromide, iodide, tetrachlorozincate, methylsulfate, trifluoromethanesulfonate, tetrafluoroborate, isatin, 1-methylisatin, 1-allylisatin, 1-hydroxymethylisatin, 5-chloroisatin, 5-methoxyisatin, 5-nitroisatin, 6-nitroisatin, 5-sulfoisatin, 5-carboxyisatin, quinisatin, 1-methylquinisatin, as well as any desired mixtures of the above-stated compounds.

Benzaldehyde, cinnamaldehyde, and naphthaldehyde as well as the derivatives thereof, in particular with one or more hydroxy, alkoxy or amino substituents are very particularly preferably used in the agents according to the invention as the reactive carbonyl compound. The compounds according to the formula I (Ca-1) are in turn here preferred,

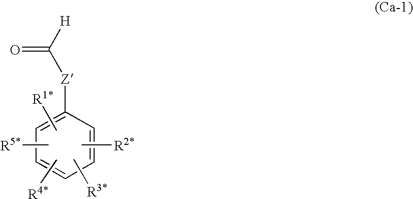

in which

R¹*, R²* and R³* mutually independently denote a hydrogen atom, a halogen atom, a C₁-C₆ alkyl group, a hydroxy group, a C₁-C₆ alkoxy group, a C₁-C₆ dialkylamino group, a di(C₂-C₆-hydroxyalkyl)amino group, a di(C₁-C₆-alkoxy-C₁-C₆-alkyl)amino group, a C₁-C₆ hydroxyalkyloxy group, a sulfonyl group, a carboxy group, a sulfonic acid group, a sulfonamido group, a sulfonamide group, a carbamoyl group, a C₂-C₆ acyl group or a nitro group,

Z′ denotes a direct bond or a vinylene group,

R⁴* and R⁵* denote a hydrogen atom or together form, with the remainder of the molecule, a 5- or 6-membered aromatic or aliphatic ring.

The benzaldehyde, naphthaldehyde or cinnamaldehyde derivatives of the component C reactive carbonyl compound are particularly preferably selected from among specific aldehydes. Agents which are preferred according to the invention are those which additionally contain at least one reactive carbonyl compound which is selected from the group consisting of 4-hydroxy-3-methoxybenzaldehyde, 3, 5-dimethoxy-4-hydroxybenzaldehyde, 4-hydroxy-1-naphthaldehyde, 4-hydroxy-2-methoxybenzaldehyde, 3,4-dihydroxy-5-methoxybenzaldehyde, 3,4,5-trihydroxybenzaldehyde, 3,5-dibromo-4-hydroxybenzaldehyde, 4-hydroxy-3-nitrobenzaldehyde, 3-bromo-4-hydroxybenzaldehyde, 4-hydroxy-3-methylbenzaldehyde, 3,5-dimethyl-4-hydroxybenzaldehyde, 5-bromo-4-hydroxy-3-methoxybenzaldehyde, 4-diethylamine-2-hydroxybenzaldehyde, 4-dimethylamino-2-methoxybenzaldehyde, coniferyl aldehyde, 2-methoxybenzaldehyde, 3-methoxybenzaldehyde, 4-methoxybenzaldehyde, 2-ethoxybenzaldehyde, 3-ethoxybenzaldehyde, 4-ethoxybenzaldehyde, 4-hydroxy-2,3-dimethoxybenzaldehyde, 4-hydroxy-2,5-dimethoxybenzaldehyde, 4-hydroxy-2,6-dimethoxybenzaldehyde, 4-hydroxy-2-methylbenzaldehyde, 4-hydroxy-2,3-dimethylbenzaldehyde, 4-hydroxy-2,5-dimethylbenzaldehyde, 4-hydroxy-2,6-dimethylbenzaldehyde, 3,5-diethoxy-4-hydroxybenzaldehyde, 2,6-diethoxy-4-hydroxybenzaldehyde, 3-hydroxy-4-methoxybenzaldehyde, 2-hydroxy-4-methoxybenzaldehyde, 2-ethoxy-4-hydroxybenzaldehyde, 3-ethoxy-4-hydroxybenzaldehyde, 4-ethoxy-2-hydroxybenzaldehyde, 4-ethoxy-3-hydroxybenzaldehyde, 2,3-dimethoxybenzaldehyde, 2,4-dimethoxybenzaldehyde, 2,5-dimethoxybenzaldehyde, 2,6-dimethoxybenzaldehyde, 3,4-dimethoxybenzaldehyde, 3,5-dimethoxybenzaldehyde, 2,3,4-trimethoxybenzaldehyde, 2,3,5-trimethoxybenzaldehyde, 2,3,6-trimethoxybenzaldehyde, 2,4,6-trimethoxybenzaldehyde, 2,4,5-trimethoxybenzaldehyde, 2,5,6-trimethoxybenzaldehyde, 2-hydroxybenzaldehyde, 3-hydroxybenzaldehyde, 4-hydroxybenzaldehyde, 2,3-dihydroxybenzaldehyde, 2,4-dihydroxybenzaldehyde, 2,4-dihydroxy-3-methylbenzaldehyde, 2,4-dihydroxy-5-methylbenzaldehyde, 2,4-dihydroxy-6-methylbenzaldehyde, 2,4-dihydroxy-3-methoxybenzaldehyde, 2,4-dihydroxy-5-methoxybenzaldehyde, 2,4-dihydroxy-6-methoxybenzaldehyde, 2,5-dihydroxybenzaldehyde, 2,6-dihydroxybenzaldehyde, 3,4-dihydroxybenzaldehyde, 3,4-dihydroxy-2-methylbenzaldehyde, 3,4-dihydroxy-5-methylbenzaldehyde, 3,4-dihydroxy-6-methylbenzaldehyde, 3,4-dihydroxy-2-methoxybenzaldehyde, 3,5-dihydroxybenzaldehyde, 2,3,4-trihydroxybenzaldehyde, 2,3,5-trihydroxybenzaldehyde, 2, 3,6-trihydroxybenzaldehyde, 2,4,6-trihydroxybenzaldehyde, 2,4,5-trihydroxybenzaldehyde, 2,5,6-trihydroxybenzaldehyde, 4-dimethylaminobenzaldehyde, 4-diethylaminobenzaldehyde, 4-dimethylamino-2-hydroxybenzaldehyde, 4-pyrrolidinobenzaldehyde, 4-morpholinobenzaldehyde, 2-morpholinobenzaldehyde, 4-piperidinobenzaldehyde, 3,5-dichloro-4-hydroxybenzaldehyde, 4-hydroxy-3,5-diiodobenzaldehyde, 3-chloro-4-hydroxybenzaldehyde, 5-chloro-3,4-dihydroxybenzaldehyde, 5-bromo-3,4-dihydroxybenzaldehyde, 3-chloro-4-hydroxy-5-methoxybenzaldehyde, 4-hydroxy-3-iodo-5-methoxybenzaldehyde, 2-methoxy-1-naphthaidehyde, 4-methoxy-1-naphthaldehyde, 2-hydroxy-1-naphthaldehyde, 2,4-dihydroxy-1-napthaldehyde, 4-hydroxy-3-methoxy-1-naphthaldehyde, 2-hydroxy-4-methoxy-1-naphthaldehyde, 3-hydroxy-4-methoxy-1-naphthaldehyde, 2,4-dimethoxy-1-naphthaldehyde, 3,4-dimethoxy-1-naphthaldehyde, 4-dimethylamino-1-naphthaldehyde, 2-nitrobenzaldehyde, 3-nitrobenzaldehyde, 4-nitrobenzaldehyde, 4-methyl-3-nitrobenzaldehyde, 3-hydroxy-4-nitrobenzaldehyde, 5-hydroxy-2-nitrobenzaldehyde, 2-hydroxy-5-nitrobenzaldehyde, 2-hydroxy-3-nitrobenzaldehyde, 2-fluoro-3-nitrobenzaldehyde, 3-methoxy-2-nitrobenzaldehyde, 4-chloro-3-nitrobenzaldehyde, 2-chloro-6-nitrobenzaldehyde, 5-chloro-2-nitrobenzaldehyde, 4-chloro-2-nitrobenzaldehyde, 2,4-dinitrobenzaldehyde, 2,6-dinitrobenzaldehyde, 2-hydroxy-3-methoxy-5-nitrobenzaldehyde, 4,5-dimethoxy-2-nitrobenzaldehyde, 6-nitropiperonal, 2-nitropiperonal, 5-nitrovanillin, 2,5-dinitrosalicylaldehyde, 5-bromo-3-nitrosalicylaldehyde, 4-nitro-1-naphthaldehyde, 2-nitrocinnamaldehyde, 3-nitrocinnamaldehyde, 4-nitrocinnamaldehyde, 4-dimethylaminocinnamaldehyde, 2-dimethylaminobenzaldehyde, 2-chloro-4-dimethylaminobenzaldehyde, 4-dimethylamino-2-methylbenzaldehyde, 4-diethylaminocinnamaldehyde, 4-dibutylaminobenzaldehyde, 4-diphenylaminobenzaldehyde, 4-(1-imidazolyl)-benzaldehyde and piperonal.

Agents which are preferred according to the invention are characterized in they are formulated as hair dyes which, relative to the weight thereof, contain 0.01 to 20 wt. %, preferably 0.05 to 15 wt. %, particularly preferably 0.1 to 12.5 wt. % and in particular 0.2 to 10 wt. % of one or more reactive carbonyl compound(s).

CH-acidic compounds are in general considered to be those which bear a hydrogen atom attached to an aliphatic carbon atom, the corresponding carbon-hydrogen bond being activated due to electron-attracting substituents. According to the invention, CH-acidic compounds also include enamines which arise by alkaline treatment of quaternized N-heterocycles with a CH-acidic alkyl group in conjugation with the quaternary nitrogen.

The CH-acidic component B compounds are preferably selected from the group consisting of 1,2,3,3-tetramethyl-3H-indolium iodide, 1,2,3,3-tetramethyl-3H-indolium p-toluenesulfonate, 1,2,3,3-tetramethyl-3H-indolium methanesulfonate, 1,3,3-trimethyl-2-methyleneindoline (Fischer's base), 2,3-dimethylbenzothiazolium iodide, 2,3-dimethylbenzothiazolium p-toluenesulfonate, 2,3-dimethylnaphtho[1,2-d]thiazolium p-toluenesulfonate, 3-ethyl-2-methylnaphtho[1,2-d]thiazolium p-toluenesulfonate, rhodanine, rhodanine-3-acetic acid, 1,4-dimethylquinolinium iodide, 1,2-dimethylquinolinium iodide, barbituric acid, thiobarbituric acid, 1,3-dimethylthiobarbituric acid, 1,3-diethylthiobarbituric acid, 1,3-diethylbarbituric acid, oxindole, 3-indoxyl acetate, 2-coumaranone, 5-hydroxy-2-coumaranone, 6-hydroxy-2-coumaranone, 3-methyl-1-phenylpyrazolin-5-one, indan-1,2-dione, indan-1,3-dione, indan-1-one, benzoylacetonitrile, 3-dicyanomethyleneindan-1-one, 2-amino-4-imino-1,3-thiazoline hydrochloride, 5,5-dimethylcyclohexane-1,3-dione, 2H-1,4-benzoxazin-4H-3-one, 3-ethyl-2-methylbenzoxazolium iodide, 3-ethyl-2-methyl-benzothiazolium iodide, 1-ethyl-4-methyl-quinolinium iodide, 1-ethyl-2-methylquinolinium iodide, 1,2,3-trimethylquinoxalinium iodide, 3-ethyl-2-methyl-benzoxazolium p-toluenesulfonate, 3-ethyl-2-methylbenzothiazolium p-toluenesulfonate, 1-ethyl-4-methylquinolinium p-toluenesulfonate, 1-ethyl-2-methylquinolinium p-toluenesulfonate, and 1,2,3-trimethylquinoxalinium p-toluenesulfonate.

Suitable component B compounds with a primary or secondary amino group are for example primary aromatic amines such as N,N-dimethyl-, N,N-diethyl-, N-(2-hydroxyethyl)-N-ethyl-, N,N-bis-(2-hydroxyethyl)-, N-(2-methoxyethyl-), 2,3-, 2,4-, 2,5-dichloro-p-phenylenediamine, 2-chloro-p-phenylenediamine, 2,5-dihydroxy-4-morpholinoaniline dihydrobromide, 2-, 3-, 4-aminophenol, 2-aminomethyl-4-aminophenol, 2-hydroxymethyl-4-amino-phenol, o-, p-phenylenediamine, o-tolylenediamine, 2,5-diaminotoluene, -phenol, -phenethol, 4-amino-3-methylphenol, 2-(2,5-diaminophenol)ethanol, 2,4-diaminophenoxyethanol, 2-(2,5-diaminophenoxy)ethanol, 4-methylamino-, 3-amino-4-(2-hydroxyethyloxy)-, 3,4-methylenediamino-, 3,4-methylene-dioxyaniline, 3-amino-2,4-dichloro-, 4-methylamino-, 2-methyl-5-amino-, 3-methyl-4-amino-, 2-methyl-5-(2-hydroxyethylamino)-, 6-methyl-3-amino-2-chloro-, 2-methyl-5-amino-4-chloro-, 3,4-methylenedioxy-, 5-(2-hydroxy-ethylamino)-4-methoxy-2-methyl-, 4-amino-2-hydroxymethylphenol, 1,3-diamino-2,4-dimethoxybenzene, 2-, 3-, 4-aminobenzoic acid, -phenylacetic acid, 2,3-, 2,4-, 2,5-, 3,4-, 3,5-diaminobenzoic acid, 4-, 5-aminosalicylic acid, 3-amino-4-hydroxy-, 4-amino-3-hydroxybenzoic acid, 2-, 3-, 4-amino-benzenesulfonic acid, 3-amino-4-hydroxybenzenesulfonic acid, 4-amino-3-hydroxynaphthalene-1-sulfonic acid, 6-amino-7-hydroxynaphthalene-2-sulfonic acid, 7-amino-4-hydroxynaphthalene-2-sulfonic acid, 4-amino-5-hydroxy-naphthalene-2,7-disulfonic acid, 3-amino-2-naphthoic acid, 3-aminophthalic acid, 5-aminoisophthalic acid, 1,3,5-, 1,2,4-triaminobenzene, 1,2,4,5-tetraaminobenzene, 2,4,5-triaminophenol, pentaaminobenzene, hexaaminobenzene, 2,4,6-triaminoresorcinol, 4,5-diaminopyrocatechol, 4,6-diaminopyrogallol, 3,5-diamino-4-hydroxypyrocatechol, aromatic anilines or phenols with a further aromatic residue, as are shown in the formula below

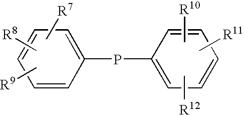

in which
R⁷ denotes a hydroxy or an amino group, which may be substituted by C₁₋₄ alkyl, C₁₋₄ hydroxyalkyl, alkoxy or C₁₋₄-alkoxy-C₁₋₄-alkyl groups,
R⁸, R⁹, R¹⁰, R¹¹ and R¹² mutually independently denote a hydrogen atom, a hydroxy or an amino group which may be substituted by C₁-C₄ alkyl, C₁-C₄ hydroxyalkyl, C₁-C₄ alkoxy, C₁-C₄ aminoalkyl or C₁-C₄-alkoxy-C₁-C₄-alkyl groups, and
P denotes a direct bond, a saturated or unsaturated carbon chain with 1 to 4 carbon atoms optionally substituted by hydroxy groups, a carbonyl, sulfoxy, sulfonyl or imino group, an oxygen or sulfur atom, or a group of the formula

-Q′-(CH₂-Q-CH₂-Q″)ₒ-

in which
Q means a direct bond, a CH₂ or CHOH group,
Q′ and Q″ mutually independently denote an oxygen atom, an NR¹³ group, in which R¹³ means a hydrogen atom, a C₁₋₄ alkyl or a hydroxy-C₁₋₄-alkyl group, the two groups possibly forming a 5-, 6- or 7-membered ring together with the residual molecule, the group O—(CH₂)ₚ—NH or NH—(CH₂)ₚ—O, in which p and p′ are 2 or 3 and
o means a number from 1 to 4,
   such as for example 4,4′-diaminostilbene, 4,4′-diaminostilbene-2,2′-disulfonic acid mono- or disodium salt, 4-amino-4′-dimethylaminostilbene, 4,4′-diaminodiphenylmethane, sulfide, sulfoxide, -amine, 4,4′-diaminodiphenylamine-2-sulfonic acid, 4,4′-diaminobenzophenone, diphenyl ether, 3,3′,4,4′-tetraaminodiphenyl, 3,3′,4,4′-tetraaminobenzophenone, 1,3-bis-(2,4-diaminophenoxy)propane, 1,8-bis-(2,5-diaminophenoxy)-3,6-dioxaoctane, 1,3-bis-(4-aminophenylamino)-propane, -2-propanol, 1,3-bis-[N-(4-aminophenyl)-2-hydroxyethylamino]-2-propanol, N,N-bis-[2-(4-aminophenoxy)-ethyl]methylamine, N-phenyl-1,4-phenylenediamine.

The above-stated compounds may be used both in free form and in the form of the physiologically acceptable salts thereof, in particular as salts of inorganic acids, such as hydrochloric or sulfuric acid.

Suitable nitrogenous heterocyclic compounds are for example 2-aminopyridine, 3-aminopyridine, 4-aminopyridine, 2-amino-3-hydroxypyridine, 2,6-diaminopyridine, 2,5-diaminopyridine, 2-(aminoethylamino)-5-amino-pyridine, 2,3-diaminopyridine, 2-dimethylamino-5-aminopyridine, 2-methylamino-3-amino-6-methoxypyridine, 2,3-diamino-6-methoxypyridine, 2,6-dimethoxy-3,5-diaminopyrimidine, 2,4,5-triaminapyridine, 2,6-dihydroxy-3,4-dimethylpyridine, N-[2-(2,4-diaminophenyl)aminoethyl]-N-(5-amino-2-pyridyl)amine, N-[2-(4-aminophenyl)aminoethyl]-N-(5-amino-2-pyridyl)amine, 2,4-dihydroxy-5,6-diaminopyrimidine, 4,5,6-triaminopyrimidine, 4-hydroxy-2,5,6-triaminopyrimidine, 2-hydroxy-4,5,6-triaminopyrimidine, 2,4,5,6-tetraaminopyrimidine, 2-methylamino-4,5,6-triaminopyrimidine, 2,4-diaminopyrimidine, 4,5-diaminopyrimidine, 2-amino-4-methoxy-6-methylpyrimidine, 3,5-diaminopyrazole, 3-aminopyrazole, 3-amino-5-hydroxypyrazole, 1-phenyl-4,5-diaminopyrazole, 1-(2-hydroxyethyl)-4,5-diaminopyrazole, 1-phenyl-3-methyl-4,5-diaminopyrazole, 4-amino-2,3-dimethyl-1-phenyl-3-pyrazolin-5-one (4-aminoantipyrine), 1-phenyl-3-methylpyrazol-5-one, 2-aminoquinoline, 3-aminoquinoline, 8-aminoquinoline, 4-aminoquinaldine, 2-aminonicotinic acid, 6-aminonicotinic acid, 5-aminoisoquinoline, 5-amino-indazole, 6-aminoindazole, 5-aminobenzimidazole, 7-aminobenzimidazole, 5-aminobenzothiazole, 7-aminobenzothiazole, 2,5-dihydroxy-4-morpholinoaniline as well as indole and indoline derivatives, such as 4-amino-indole, 5-aminoindole, 6-aminoindole, 7-aminoindole, 5,6-dihydroxyindole, 5,6-dihydroxyindoline and 4-hydroxyindoline. The above-stated compounds may be used both in free form and in the form of the physiologically acceptable salts thereof, for example as salts of inorganic acids, such as hydrochloric or sulfuric acid,

Amino acids which may preferably be considered are all naturally occurring and synthetic alpha-amino acids, for example the amino acids obtainable by hydrolysis from plant or animal proteins, for example collagen, keratin, casein, elastin, soy protein, wheat gluten or almond protein. Both those amino acids which exhibit an acidic reaction and those which exhibit an alkaline reaction may be used. Preferred amino acids are arginine, histidine, tyrosine, phenylalanine, DOPA (dihydroxyphenylalanine), ornithine, lysine and tryptophan. Other amino acids may, however, also be used, such as for example 6-aminocaproic acid,

The oligopeptides may here be not only naturally occurring or synthetic oligopeptides, but also oligopeptides present in polypeptide or protein hydrolysates, providing that they have sufficient water solubility for use in the dyes according to the invention. Examples which may be mentioned are for example glutathione, or the oligopeptides present in the hydrolysates of collagen, keratin, casein, elastin, soy protein, wheat gluten or almond protein. Use in conjunction with compounds having a primary or secondary amino group or with aromatic hydroxy compounds is here preferred.

Suitable aromatic hydroxy compounds are for example 2-methylresorcinol, 4-methylresorcinol, 5-methylresorcinol, 2,5-dimethylresorcinol, resorcinol, 3-methoxyphenol, pyrocatechol, hydroquinone, pyrogallol, phloroglucinol, hydroxyhydroquinone, 2-methoxyphenol, 3-methoxyphenol, 4-methoxyphenol, 3-dimethylaminophenol, 2-(2-hydroxyethyl)phenol, 3,4-methylenedioxyphenol, 2,4-dihydroxybenzoic acid, 3,4-dihydroxybenzoic acid, 2,4-dihydroxyphenylacetic acid, 3,4-dihydroxyphenylacetic acid, gallic acid, 2,4,6-trihydroxybenzoic acid, 2,4,6-trihydroxyacetophenone, 2-chlororesorcinol, 4-chlororesorcinol, 1-naphthol, 1,5-dihydroxynaphthalene, 2,3-dihydroxynaphthalene, 2,7-dihydroxynaphthalene, 6-dimethylamino-4-hydroxy-2-naphthalenesulfonic acid and 3,6-dihydroxy-2,7-naphthalenesulfonic acid.

CH-active compounds which may be stated by way of example are 1,2,3,3-tetramethyl-3H-indolium iodide, 1,2,3,3-tetramethyl-3H-indolium p-toluenesulfonate, 1,2,3,3-tetramethyl-3H-indolium methanesulfonate, Fischer's base (1,3,3-trimethyl-2-methyleneindoline), 2,3-dimethylbenzothiazolium iodide, 2,3-dimethylbenzothiazolium p-toluenesulfonate, rhodanine, rhodanine-3-acetic acid, 1-ethyl-2-quinaldinium iodide, 1-methyl-2-quinaldinium iodide, barbituric acid, thiobarbituric acid, 1,3-dimethylthiobarbituric acid, diethylthiobarbituric acid, oxindole, 3-indoxyl acetate, coumaranone and 1-methyl-3-phenyl-2-pyrazolinone.

The CH-acidic compounds are preferably selected from the formulae (II) and/or (Ill) and/or (IV)

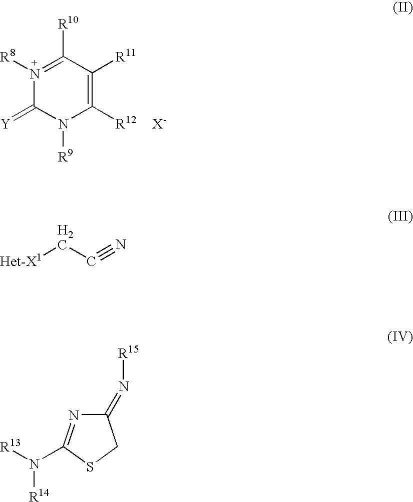

in which
R⁸ and R⁹ mutually independently denote a linear or cyclic C₁-C₆ alkyl group, a C₂-C₆ alkenyl group, an optionally substituted aryl group, an optionally substituted heteroaryl group, an aryl-C₁-C₆-alkyl group, a C₁-C₆-hydroxyalkyl group, a C₂-C₆ polyhydroxyalkyl group, a C₁-C₆-alkoxy-C₁-C₆-alkyl group, a group R^{I}R^{II}N—(CH₂)ₘ—, in which R^{I} and R^{II} mutually independently denote a hydrogen atom, a C₁-C₄ alkyl group, a C₁-C₄ hydroxyalkyl group or an aryl-C₁-C₆-alkyl group, R^{I} and R^{II}, together with the nitrogen atom, being capable of forming a 5-, 6- or 7-membered ring and m denoting a number 2, 3, 4, 5 or 6,
R¹⁰ and R¹² mutually independently denote a hydrogen atom or a C₁-C₆ alkyl group, at least one of the residues R¹⁰ and R¹² meaning a C₁-C₆ alkyl group,
R¹¹ denotes a hydrogen atom, a C₁-C₆ alkyl group, a C₁-C₆ hydroxyalkyl group, a C₂-C₆ polyhydroxyalkyl group, a C₁-C₆ alkoxy group, a C₁-C₆ hydroxyalkoxy group, a group R^{III}R^{IV}N—(CH₂)_{q}—, in which R^{III} and R^{IV} mutually independently denote a hydrogen atom, a C₁-C₆ alkyl group, a C₁-C₆ hydroxyalkyl group or an aryl-C₁-C₆-alkyl group and q denotes a number 1, 2, 3, 4, 5 or 6, the residue R¹¹, together with one of the residues R¹⁰ or R¹², being capable of forming a 5- or 6-membered aromatic ring which may optionally be substituted with a halogen atom, a C₁-C₆ alkyl group, a C₁-C₆ hydroxyalkyl group, a C₂-C₆ polyhydroxyalkyl group, a C₁-C₆ alkoxy group, a C₁-C₆ hydroxyalkoxy group, a nitro group, a hydroxy group, a group R^{V}R^{VI}N(CFH₂)ₛ—, in which R^{V} and R^{VI} mutually independently denote a hydrogen atom, a C₁-C₆ alkyl group, a C₁-C₆ hydroxyalkyl group or an aryl-C₁-C₆-alkyl group and s denotes a number 0, 1, 2, 3, 4, 5 or 6,
R¹³ and R¹⁴ either form, together with the nitrogen atom, a saturated or unsaturated 5- or 6-membered ring or mutually independently denote a (C₁-C₆) alkyl group, a (C₂-C₆) alkenyl group, an aryl group, an aryl-(C₁-C₆) alkyl group, a (C₂-C₆) hydroxyalkyl group, a (C₂-C₆) polyhydroxyalkyl group or a group R^{I}R^{II}N—(CH₂)ₘ—, in which R^{I} and R^{II} mutually independently denote a hydrogen atom, a (C₁-C₆) alkyl group, a (C₁-C₆) alkenyl group or an aryl-C₁-C₆-alkyl group, R^{I} and R^{II}, together with the nitrogen atom, being capable of forming a 5-, 6- or 7-membered ring and m denoting a number 2, 3, 4, 5 or 6, and
R¹⁵ means a hydrogen atom, a (C₁-C₆) alkyl group, a (C₂-C₆) alkenyl group, an aryl group, an aryl-(C₁-C₆)-alkyl group, a (C₂-C₆) hydroxyalkyl group, a (C₂-C₆) polyhydroxyalkyl group or a group R^{III}R^{IV}N—(CH₂)ₙ—, in which R^{III} and R^{IV} mutually independently denote a hydrogen atom, a (C₁-C₆) alkyl group, a (C₁-C₆) alkenyl group or an aryl-C₁-C₆-alkyl group, R^{III} and R^{IV}, together with the nitrogen atom, being capable of forming a 5-, 6- or 7-membered ring and n denoting a number 2, 3, 4, 5 or 6
Y denotes an oxygen atom, a sulfur atom or a group NR^{VII}, in which R^{VII} denotes a hydrogen atom, an aryl group, a heteroaryl group, a C₁-C₆ alkyl group or a C₁-C₆ arylalkyl group,
X⁻ denotes a physiologically acceptable anion,
Het denotes an optionally substituted heteroaromatic,
X¹ denotes a direct bond or a carbonyl group.

The enamine forms of the compounds of the formula II have an equivalent action. Reference is here explicitly made to the full disclosure of document WO-A1-2004/022016.

At least one group R¹⁰ or R¹² according to the formula II mandatorily denotes a C₁-C₆ alkyl group. This alkyl group preferably bears at least two hydrogen atoms on its alpha carbon atom. Particularly preferred alkyl groups are the methyl, ethyl, propyl, n-butyl, iso-butyl, n-pentyl, neopentyl, n-hexyl group. R¹⁰ and R¹² very particularly preferably mutually independently denote hydrogen or a methyl group, at least one group R¹⁰ or R¹² meaning a methyl group.

In a preferred embodiment, Y denotes an oxygen or a sulfur atom, particularly preferably an oxygen atom.

The residue R⁸ is, preferably selected from a (C₁-C₆) alkyl group (particularly preferably a methyl group), a C₂-C₆ alkenyl group (in particular an allyl group), a hydroxy-(C₂ to C₆)-alkyl group, in particular a 2-hydroxyethyl group, or an optionally substituted benzyl group.

R¹¹ preferably denotes a hydrogen atom.

The residues R⁹, R¹⁰ and R¹² particularly preferably denote a methyl group, the residue R¹¹ denotes a hydrogen atom, Y an oxygen or a sulfur atom and the residue R⁸ is selected from a (C₁-C₆) alkyl group (particularly preferably a methyl group), a C₂-C₆ alkenyl group (in particular an allyl group), a hydroxy-(C₂ to C₆)-alkyl group, in particular a 2-hydroxyethyl group, or an optionally substituted benzyl group.

The compounds according to the formula II are preferably selected from one or more compounds of the group of salts with a physiologically acceptable counterion X⁻, which is formed of salts of 1,2-dihydro-1,3,4,6-tetramethyl-2-oxopyrimidinium, 1,2-dihydro-1,3-diethyl-4,6-dimethyl-2-oxopyrimidinium, 1,2-dihydro-1,3-dipropyl-4,6-dimethyl-2-oxopyrimidinium, 1,2-dihydro-1,3-di(2-hydroxyethyl)-4,6-dimethyl-2-oxopyrimidinium, 1,2-dihydro-1,3-diphenyl-4,6-dimethyl-2-oxopyrimidinium, 1,2-dihydro-1,3,4-trimethyl-2-oxopyrimidinium, 1,2-dihydro-1,3-diethyl-4-methyl-2-oxopyrimidinium, 1,2-dihydro-1,3-dipropyl-4-methyl-2-oxopyrimidinium, 1,2-dihydro-1,3-di(2-hydroxyethyl)-4-methyl-2-oxopyrimidinium, 1,2-dihydro-1,3-diphenyl-4-methyl-2-oxopyrimidinium, 1-allyl-1,2-dihydro-3,4,6-trimethyl-2-oxopyrimidinium, 1,2-dihydro-1-(2-hydroxyethyl)-3,4,6-trimethyl-2-oxopyrimidinium, 1,2-dihydro-1,3,4,6-tetramethyl-2-thioxopyrimidinium, 1,2-dihydro-1,3-diethyl-4,6-dimethyl-2-thioxopyrimidinium, 1,2-dihydro-1,3-dipropyl-4,6-dimethyl-2-thioxopyrimidinium, 1,2-dihydro-1,3-di(2-hydroxyethyl)-4,6-dimethyl-2-thioxopyrimidinium, 1,2-dihydro-1,3-diphenyl-4,6-dimethyl-2-thioxopyrimidinium, 1,2-dihydro-1,3,4-trimethyl-2-thioxopyrimidinium, 1,2-dihydro-1,3-diethyl-4-methyl-2-thioxopyrimidinium, 1,2-dihydro-1,3-dipropyl-4-methyl-2-thioxopyrimidinium, 1,2-dihydro-1,3-di(2-hydroxyethyl)-4-methyl-2-thioxopyrimidinium, 1,2-dihydro-1,3-diphenyl-4-methyl-2-thioxopyrimidinium, 1,2-dihydro-3,4-dimethyl-2-oxoquinazolinium and 1,2-dihydro-3,4-dimethyl-2-thioxoquinazolinium.

Very particularly preferred agents according to the invention are characterized in that they contain as the CH-acidic compound salts of 1,2-dihydro-1,3,4,6-tetramethyl-2-oxopyrimidinium, salts of 1,2-dihydro-1,3,4-trimethyl-2-oxopyrimidinium, salts of 1,2-dihydro-1,3,4,6-tetramethyl-2-thioxopyrimidinium, salts of 1-allyl-1,2-dihydro-3,4,6-trimethyl-2-oxopyrimidinium, salts of 1,2-dihydro-1-(2-hydroxyethyl)-3,4,6-trimethyl-2-oxo-pyrimidinium, 2-(cyanomethyl)benzimidazole, 4,5-dihydro-4-imino-2-(1-piperidinyl)-thiazole and/or the hydrochloride thereof, 4,5-dihydro-4-imino-2-(4-morpholinyl)-thiazole and/or the hydrochloride thereof, 4,5-dihydro-4-imino-2-(1-pyrrolidinyl)-thiazole and/or the hydrochloride thereof.
X⁻ in the formula (II) as well as in the above lists preferably denotes halide, benzenesulfonate, p-toluenesulfonate, C₁-C₄ alkane sulfonate, trifluoromethanesulfonate, perchlorate, 0.5 sulfate, hydrogensulfate, tetrafluoroborate, hexafluorophosphate or tetrachiorozincate. The anions chloride, bromide, iodide, hydrogensulfate or p-toluenesulfonate are particularly preferably used as X⁻.

The residue Het according to the formula (III) preferably denotes the molecular fragment of the formula (V),

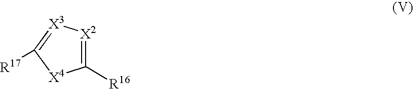

in which
R¹⁶ and R¹⁷ mutually independently denote a hydrogen atom, a hydroxy group, a halogen atom, a nitro group, a linear or cyclic C₁-C₆ alkyl group, a C₂-C₆ alkenyl group, an optionally substituted aryl group, a cyanomethyl group, a cyanomethylcarbonyl group, an optionally substituted heteroaryl group, an aryl-C₁-C₆-alkyl group, a C₁-C₆ hydroxy-alkyl group, a C₂-C₆ polyhydroxyalkyl group, a C₁-C₆ alkoxy group, a C₁-C₆ alkoxycarbonyl group, a C₁-C₆-alkoxy-C₂-C₆-alkyl group, a C₁-C₆ sulfoalkyl group, a C₁-C₆ carboxyalkyl group, a group R^{VIII}R^{IX}N—(CH₂)ₘ—, in which R^{VIII} and R^{IX} mutually independently denote a hydrogen atom, a linear or cyclic C₁-C₆ alkyl group, a C₂-C₆ alkenyl group, a C₁-C₆ hydroxyalkyl group, or an aryl-C₁-C₄-alkyl group, R^{VIII} and R^{IX}, together with the nitrogen atom, being capable of forming a 5-, 6- or 7-membered ring and m denoting a number 0, 1, 2, 3, or 4,
   R¹⁶ and/or R¹⁷ being capable of forming an optionally substituted aromatic or heteroaromatic 5- or 6-membered ring fused to the ring of the remainder of the molecule,
X² and X³ mutually independently denote a nitrogen atom or a group CR¹⁵, R¹⁵ denoting a hydrogen atom, a hydroxy group, a halogen atom, a nitro group, a linear or cyclic C₁-C₆ alkyl group, a C₂-C₆ alkenyl group, an optionally substituted aryl group, a cyanomethyl group, a cyanomethylcarbonyl group, an optionally substituted heteroaryl group, an aryl-C₁-C₆-alkyl group, a C₁-C₆ hydroxyalkyl group, a C₂-C₆ polyhydroxyalkyl group, a C₁-C₆ alkoxy group, a C₁-C₆ alkoxycarbonyl group, a C₁-C₆-alkoxy-C₂-C₆-alkyl group, a C₁-C₆ sulfoalkyl group, a C₁-C₆ carboxyalkyl group and a group R^{X}R^{XI}N—(CH₂)ₙ—, in which R^{X} and R^{XI} mutually independently denote a hydrogen atom, a linear or cyclic C₁-C₆ alkyl group, a C₂-C₆ alkenyl group, a C₁-C₆ hydroxyalkyl group or an aryl-C₁-C₄-alkyl group, R^{X} and R^{XI}, together with the nitrogen atom, being capable of forming a 5-, 6- or 7-membered ring and n denoting a number 0, 1, 2, 3 or 4,
   at least one of the substituents X² and X³, together with the remainder of the molecule, being capable of forming a fused, optionally substituted aromatic 5- or 6-membered ring,
X⁴ denotes an oxygen atom, a sulfur atom, a vinylene group or a group N—H, the latter two stated groups mutually independently optionally being capable of substitution with a linear or cyclic C₁-C₆ alkyl group, a C₂-C₆ alkenyl group, an optionally substituted aryl group, an optionally substituted heteroaryl group, an aryl-C₁-C₆-alkyl group, a C₂-C₆ hydroxy-alkyl group, a C₂-C₆ polyhydroxyalkyl group, a C₁-C₆-alkoxy-C₂-C₆-alkyl group, a C₁-C₆ sulfoalkyl group, a C₁-C₆ carboxyalkyl group, a group R^{XII}RX^{III}N—(CH₂)ₚ—, in which R^{XII} and R^{XIII} mutually independently denote a hydrogen atom, a linear or cyclic C₁-C₆ alkyl group, a C₂-C₅ alkenyl group, a C₁-C₆ hydroxyalkyl group, or an aryl-C₁-C₄-alkyl group, R^{XII} and R^{XIII}, together with the nitrogen atom, being capable of forming a 5-, 6- or 7-membered ring and p denoting a number 0, 1, 2, 3 or 4,
   providing that, if X⁴ denotes a vinylene group, at least one of the groups X² or X³ means a nitrogen atom.

Attachment of the heterocyclic ring according to the formula (V) to the molecular fragment —X¹—CH₂—C≡N, while retaining the compound according to the invention according to the formula (III), is made to the ring of the heterocycle and replaces a hydrogen atom attached to said ring. It is consequently absolutely necessary for the substituents R¹⁶, R¹⁷, X², X³ and X⁴ to be selected such that at least one of these substituents permits the formation of such a bond. It is consequently essential for at least one of the residues R¹⁶ or R¹⁷ to form the bond to the molecular fragment —X¹—CH₂—C≡N, if X⁴ is an oxygen atom or a sulfur atom and X² and X³ mean a nitrogen atom.

The residue Het according to formula (III) is particularly preferably derived from the heteroaromatics furan, thiophene, pyrrole, isoxazole, isothiazole, imidazole, oxazole, thiazole, pyridine, pyridazine, pyrimidine, pyrazine, 1,2,3-triazine, 1,2,4-triazine, 1,3,5-triazine, benzopyrrole, benzofuran, benzothiophene, benzimidazole, benzoxazole, indazole, benzoisoxazole, benzoisothiazole, indole, quinoline, isoquinoline, cinnoline, phthalazine, quinazoline, quinoxaline, acridine, benzoquinoline, benzoisoquinoline, benzothiazole, phenazine, benzocinnoline, benzoquinazoline, benzoquinoxaline, phenoxazine, phenothiazine, naphthyridine, phenanthroline, indolizine, quinolizine, carboline, purine, pteridine and coumarin, the above-stated heteroaromatics being capable of substitution with at least one group selected from a halogen atom, a nitro group, a thio group, a thio-(C₁-C₆)-alkyl group, a heteroaryl group, an aryl group, a (C₁-C₆) alkyl group, a (C₁-C₆) alkoxy group, a hydroxy group, a (C₂-C₆) hydroxyalkyl group, a (C₂-C₆) polyhydroxy-alkyl group, a (C₁-C₆)-alkoxy-(C₁-C₆)-alkyl group, an aryl-(C₁-C₆)-alkyl group, an amino group, a (C₁-C₆) monoalkylamino group, a (C₁-C₆) dialkylamino group, a dialkylaminoalkyl group —(CH₂)ₙ—NR′R″, in which n is an integer between 2 and 6 and R′ and R″ mutually independently mean a linear or branched alkyl group, which may optionally together form a ring.

The compounds according to formula (Ill) are preferably selected from the group consisting of 2-(2-furoyl)-acetonitrile, 2-(5-bromo-2-furoyl)-acetonitrile, 2-(5-methyl-2-trifluoromethyl-3-furoyl)-acetonitrile, 3-(2,5-dimethyl-3-furyl)-3-oxopropanenitrile, 2-(2-thenoyl)-acetonitrile, 2-(3-thenoyl)-acetonitrile, 2-(5-fluoro-2-thenoyl)-acetonitrile, 2-(5-chloro-2-thenoyl)-acetonitrile, 2-(5-bromo-2-thenoyl)-acetonitrile, 2-(5-methyl-2-thenoyl)-acetonitrile, 2-(2,5-dimethylpyrrol-3-oyl)-acetonitrile, 2-(1,2,5-trimethylpyrrol-3-oyl)-acetonitrile, 1H-benzimidazol-2-ylacetonitrile, 1H-benzothiazol-2-ylacetonitrile, 2-(pyrid-2-yl)-acetonitrile, 2,6-bis(cyanomethyl)-pyridine, 2-(indol-3-oyl)-acetonitrile, 2-(2-methylindol-3-oyl)-acetonitrile, 8-cyanoacetyI-7-methoxy-4-methylcoumarin, 2-(2-isopropyl-5,6-benzoquinolin-4-oyl)-acetonitrile, 2-(2-phenyl-5,6-benzoquinolin-4-oyl)-acetonitrile, 2-(quinoxalin-2-yl)-acetonitrile, 2-(coumaron-2-yl)-acetonitrile, 6,7-dichloro-5-(cyanoacetyl)-2,3-dihydro-1-benzofuran-2-carboxylic acid tert.-butyl ester, 2-(6-hydroxy-4,7-dimethoxy-1-benzofuran-5-oyl)-acetonitrile and 2-(1-phenyl-1,4-dihydrothiochromeno[4,3c]pyrazol-3-oyl)-acetonitrile. 1H-benzimidazol-2-ylacetonitrile [2-(cyanomethyl)benzimidazole] is particularly preferred.

Agents which are preferred according to the invention are characterized in that they are formulated as hair dyes which, relative to the weight thereof, contain 0.01 to 20 wt. %, preferably 0.05 to 15 wt. %, particularly preferably 0.1 to 12.5 wt. % and in particular 0.2 to 10 wt. % of one or more CH-acidic compound(s).

It has been found that making simultaneous use of azoles enhances the action of the agents according to the invention. In particular, the activation of hydrogen peroxide and the reduction of yellow coloration during blonding are further enhanced by the simultaneous use of azoles.

Agents for dyeing and/or lightening keratin fibers which are preferred according to the invention are therefore characterized in that, relative to the weight thereof, they contain 1 to 20 wt. % of at least one optionally substituted, in particular benzo-fused azole, preferably at least one optionally substituted and/or benzo-fused pyrrole and/or pyrazole and/or imidazole and/or 1,2,3-triazole and/or 1,2,4-triazole and/or 1,2,3,4-tetrazole.

The parent substances of the stated compounds are shown below:

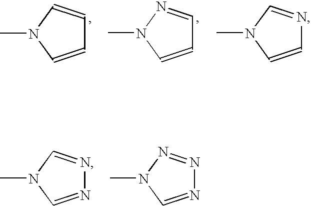

The azoles may bear a hydrogen atom or be substituted at the N atoms indicated with a bond. Aryl, alkyl or alkenyl residues have proved satisfactory as substituents on the particular N atom. The azoles may additionally be substituted or benzo-fused on the carbon atom(s). Benzo-fused azoles which are preferably usable according to the invention have the following structures, the aromatic rings optionally being capable of further substitution.

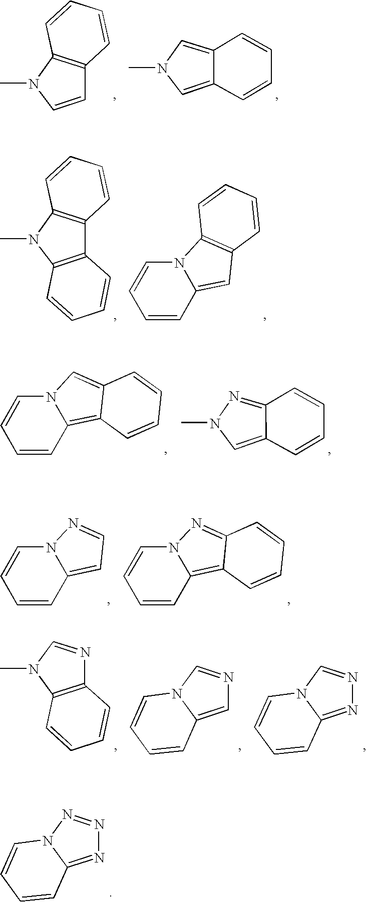

It is particularly preferred according to the invention to use pyrazoles. Agents for dyeing and/or lightening keratin fibers which are particularly preferred according to the invention are here characterized in that, relative to the weight thereof, they contain 1 to 20 wt. % of at least one imidazole compound according to formula (VI) and/or the physiologically acceptable salts thereof

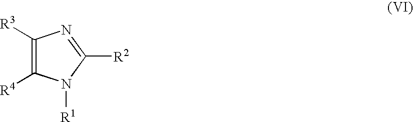

in which
R¹ denotes a hydrogen atom, an optionally substituted aryl group, or a (C₁-C₆) alkyl group,
R² denotes a hydrogen atom, a carboxaldehyde group, a (C₁-C₆) alkyl group or a nitro group,
R³ denotes a hydrogen atom, a carboxy-(C₁-C₆)-alkyl group, an amino-(C₁-C₆)-alkyl group, a carboxyl group, a carboxaldehyde group, a (C₁-C₆) alkyl group, a nitro group, a 2-amino-3-hydroxy-propyl group or a —CH₂—CH(—NH₂)—COOH group,
R⁴ denotes a hydrogen atom, a carboxaldehyde group or a carboxyl group.

The imidazole compound(s) of the formula (VI) are preferably used within relatively narrow quantity ranges. Agents for dyeing and/or lightening keratin fibers which are preferred according to the invention are here those which contain at least one imidazole compound according to formula (VI) and/or the physiologically acceptable salts thereof in quantities of 2 to 15 wt %, preferably of 2.5 to 12.5 wt. %, particularly preferably of 3 to 10 wt. % and in particular of 4 to 8 wt. %.

The imidazole compounds according to formula (VI) are preferably selected from at least one representative from a group which is formed of histamine, D-histidine, L-histidine, DL-histidine, D-histidinol, L-histidinol, DL-histidinol, imidazole, imidazole-4-acetic acid, imidazole-4-carboxylic acid, imidazole-4,5-dicarboxylic acid, imidazole-2-carboxaldehyde, imidazole-4-carboxaldehyde, imidazole-5-carboxaldehyde, 2-nitroimidazole, 4-nitroimidazole, 4-methylimidazole-5-carboxaldehyde, N-methylimidazole-2-carboxaldehyde, 4-methylimidazole, 2-methylimidazole, N-methylimidazole, N-(4-aminophenyl)imidazole, as well as the physiologically acceptable salts of the above-stated compounds. Imidazole is particularly preferably used according to the invention.

In summary, agents which are preferred according to the invention are characterized in that they contain at least one imidazole compound according to formula (VI) and/or the physiologically acceptable salts thereof in quantities of 2 to 15 wt. %, preferably of 2.5 to 12.5 wt. %, particularly preferably of 3 to 10 wt. % and in particular of 4 to 8 wt. %, preferred imidazole compounds according to formula (VI) and/or the physiologically acceptable salts thereof being selected from histamine, D-histidine, L-histidine, DL-histidine, D-histidinol, L-histidinol, DL-histidinol, imidazole, imidazole-4-acetic acid, imidazole-4-carboxylic acid, imidazole-4,5-dicarboxylic acid, imidazole-2-carboxaldehyde, imidazole-4-carboxaldehyde, imidazole-5-carboxaldehyde, 2-nitroimidazole, 4-nitroimidazole, 4-methylimidazole-5-carboxaldehyde, N-methylimidazole-2-carboxaldehyde, 4-methylimidazole, 2-methylimidazole, N-methylimidazole, N-(4-aminophenyl)-imidazole, as well as the physiologically acceptable salts of the above-stated compounds.

Since the “boosters” are readily broken down by hydrolysis, it is preferred not to add them to the oxidizing agent preparation or the application mixture until immediately before application.

The agents according to the invention may contain as an additional alkalizing agent at least one compound from among amino acids and/or oligopeptides, these in each case comprising at least two amino groups and at least one —COOH— or —SO₃H group and a 2.5% solution thereof in water in each case exhibiting a pH value of greater than 9.0.

Preferred alkalizing agents are aminocarboxylic acids, in particular α-aminocarboxylic acids and omega-aminocarboxylic acids. Among the α-aminocarboxylic acids, lysine and in particular arginine are in turn particularly preferred.

Said amino acids may preferably be added to the agents according to the invention in free form. In a range of cases, however, it is also possible to use the amino acids in salt form. Preferred salts are then compounds with hydrohalic acids, in particular hydrochlorides and hydrobromides.

The amino acids may furthermore also be used in the form of oligopeptides and protein hydrolysates, if it is ensured that the necessary quantities of the amino acids used according to the invention are contained therein.

One particularly preferred alkalizing agent is arginine.

Agents which are preferred according to the invention are characterized in that they additionally contain 0.05 to 5 wt. %, preferably 0.1 to 2.5 wt. %, particularly preferably 0.15 to 1 wt. % and in particular 0.2 to 0.5 wt. % of amino acid(s), preferably (an) amino acid(s) from the group glycine and/or alanine and/or valine and/or lysine and/or leucine and/or threonine and/or serine.

The anhydrous compositions according to the invention may additionally contain at least one further bleach booster which differs from the inorganic per-salts.

Bleach boosters which may be used are compounds which, under perhydrolysis conditions, yield aliphatic peroxycarboxylic acids with preferably 1 to 10 C atoms, in particular 2 to 4 C atoms, and/or optionally substituted per-benzoic acid. Suitable substances are those which bear O— and/or N-acyl groups having the stated number of C atoms and/or optionally substituted benzoyl groups. Preferred compounds are repeatedly acylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic anhydrides, in particular phthalic anhydride, acylated polyhydric alcohols, in particular triacetin, ethylene glycol diacetate and 2,5-diacetoxy-2,5-dihydrofuran.

In order further to increase their lightening power, anhydrous agents according to the invention may additionally contain at least one optionally hydrated SiO₂ compound as bleach booster. Although even small quantities of the optionally hydrated SiO₂ compounds increase lightening power, it may be preferred according to the invention to use the optionally hydrated SiO₂ compounds in quantities of 0.05 wt. % to 15 wt. %, particularly preferably in quantities of 0.15 wt. % to 10 wt. % and very particularly preferably in quantities of 0.2 wt. % to 5 wt. %, in each case relative to the anhydrous composition according to the invention. The stated quantities here in each case indicate the content of SiO₂ compounds (excluding their water content) in the agents.

No restrictions apply in principle to the present invention with regard to the optionally hydrated SiO₂ compounds. Silicas, the oligomers and polymers thereof as well as the salts thereof are preferred. Preferred salts are alkali metal salts, in particular the potassium and sodium salts. Sodium salts are very particularly preferred.

The optionally hydrated SiO₂ compounds may be present in various forms. According to the invention, the SiO₂ compounds are preferably used in the form of silica gels or particularly preferably as water glass. These SiO₂ compounds may in part assume the form of an aqueous solution.

Water glasses formed of silicates of the formula (SiO₂)ₙ(Na₂O)ₘ(K₂O)ₚ, n denoting a positive rational number and m and p mutually independently denoting a positive rational number or 0, providing that at least one of the parameters M or p differs from 0 and the ratio between n and the sum of m and p is between 1:4 and 4:1, are very particularly preferred according to the invention.

In addition to the components described by the empirical formula, the water glasses may contain small quantities of still further additives, such as for example phosphates or magnesium salts.

Water glasses which are particularly preferred according to the invention are distributed inter glia by Henkel under the names Ferrosil® 119, soda water glass 40/42, Portil® A, Portil® AW and Portil® W and by Akzo under the name Britesil® C20.

Agents which are preferred according to the invention are characterized in that they additionally contain 0.05 to 15 wt. %, particularly preferably 0.15 to 10 wt. % and in particular 0.2 to 5 wt. % of at least one optionally hydrated SiO₂ compound, preferred agents containing water glasses which are formed from silicates of the formula (SiO₂)ₙ(Na₂0)ₘ(K₂O)ₚ, in which n denotes a positive rational number and m and p mutually independently denote a positive rational number or 0, providing that at least one of the parameters m or p differs from 0 and the ratio between n and the sum of m and p is between 1:4 and 4:1.

In addition to the stated constituents, it has also proved preferable to use carbonates and carbonate analogs in the agents according to the invention. Agents for dyeing and/or lightening keratin fibers which are preferred according to the invention additionally contain 0.1 to 25 wt. %, preferably 0.5 to 20 wt. %, particularly preferably 1 to 15 wt. % and in particular 1.5 to 10 wt. % of at least one substance from the groups of
i. carbonates and/or
ii. monoalkyl carbonates (carbonic acid monoesters) and/or
iii. carbonic acid monoamides and/or
iv. silyl carbonates and/or
v. silyl carbamates

Carbonates which may be considered are in particular ammonium, alkali metal and alkaline earth metal carbonates and hydrogencarbonates. Sodium hydrogencarbonate, ammonium hydrogencarbonate, sodium carbonate, ammonium carbonate and ammonium carbamate are particularly preferred, for example.

In addition to or instead of carbonates, the agents according to the invention may preferably also contain at least one carbonic acid monoester and/or at least one carbonic acid monoamide. These substances are described in detail below.

Agents which are preferred according to the invention are characterized in that they contain at least one carbonic acid monoester of the formula (C-I)

R—O—C(O)—O—H (C-I)

in which R denotes a saturated or unsaturated, straight-chain, branched or cyclic, substituted or unsubstituted hydrocarbon residue, or a substituted or unsubstituted aryl group or a substituted or unsubstituted heterocycle.

In the formula (C-I), R preferably denotes a substituted or unsubstituted, straight-chain or branched alkyl, alkenyl or alkynyl residue, hydroxy, amino, nitro, sulfonic acid groups or halogens preferably being considered as substituents. Further preferred residues R are phenyl and benzyl residues as well as further substituted representatives. R particularly preferably denotes a C₁₋₆ alkyl group. Examples of C₁-C₆ alkyl groups according to the invention are the groups methyl, ethyl, n-propyl, isopropyl, n-butyl, sec-butyl, iso-butyl, tert.-butyl, pentyl, iso-pentyl and hexyl.

Agents which are particularly preferred according to the invention are characterized in that the residue R in the formula (C-I) is selected from methyl, ethyl, n-propyl, iso-propyl, n-butyl, iso-butyl, tert.-butyl as well as hydroxymethyl and hydroxyethyl residues.

Alternatively to the carbonic acid monoester or in conjunction therewith, the agents according to the invention may contain carbonic acid monoamides. Agents which are preferred according to the invention are here characterized in that they contain at least one carbonic acid monoamide of the formula (C-II)

R—NH—C(O)—O—H (C-II)

in which R denotes a saturated or unsaturated, straight-chain, branched or cyclic, substituted or unsubstituted hydrocarbon residue, or a substituted or unsubstituted aryl group or a substituted or unsubstituted heterocycle.

In the formula (C-II), R preferably denotes a substituted or unsubstituted, straight-chain or branched alkyl, alkenyl or alkynyl residue, hydroxy, amino, nitro, sulfonic acid groups or halogens preferably being considered as substituents. Further preferred residues R are phenyl and benzyl residues as well as further substituted representatives. R particularly preferably denotes a C₁₋₆ alkyl group. Examples of C₁-C₆ alkyl groups according to the invention are the groups methyl, ethyl, n-propyl, isopropyl, n-butyl, sec-butyl, iso-butyl, tert.-butyl, pentyl, iso-pentyl and hexyl.

Agents which are particularly preferred according to the invention are characterized in that the residue R in the formula (C-I) is selected from methyl, ethyl, n-propyl, iso-propyl, n-butyl, iso-butyl, tert.-butyl as well as hydroxymethyl and hydroxyethyl residues.

The acidic H atom of the carbonic acid monoester or monoamide may also assume neutralized form, i.e. salts of carbonic acid monoesters or carbonic acid monoamides may also be used according to the invention. Agents according to the invention which are preferred are here those which contain the carbonic acid monoester or the carbonic acid monoamide in entirely or partially neutralized form, preferably in the form of the alkali metal, ammonium, alkaline earth metal or aluminum salt thereof and in particular in the form of the sodium salt thereof.

Irrespective of whether the agents according to the invention contain one or more carbonic acid monoesters and/or one or more carbonic acid monoamides, agents according to the invention which are preferred are those which contain the carbonic acid monoester(s) and/or carbonic acid monoamide(s) in quantities of 0.1 to 20 wt. %, preferably of 0.5 to 18 wt. %, particularly preferably of 2 to 15 wt. % and in particular of 5 to 12 wt. %, in each case relative to the entire agent.

In addition to or instead of carbonates and/or carbonic acid monoesters and/or carbonic acid monoamides, the agents according to the invention preferably also contain at least one silyl carbonate and/or at least one silyl carbamate. These substances are described in detail below.

Agents which are preferred according to the invention are characterized in that they contain at least one silyl carbonate of the formula (C-III)

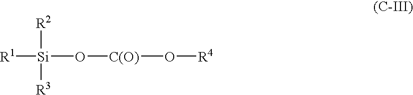

in which the residues R¹, R² and R³ mutually independently denote a hydrogen atom, a saturated or unsaturated, straight-chain, branched or cyclic, substituted or unsubstituted hydrocarbon residue or a trialkylsilyl group, preferably a trimethylsilyl group or a substituted or unsubstituted aryl group or a substituted or unsubstituted heterocycle or a halogen, a substituted or unsubstituted hydroxy, oxo, amino, imino group and the residue R⁴ denotes a chemical bond to the Si atom or to one of the residues R¹, R² or R³, a hydrogen atom, a saturated or unsaturated, straight-chain, branched or cyclic, substituted or unsubstituted hydrocarbon residue or a substituted or unsubstituted silyl or aluminyl group or a substituted or unsubstituted aryl group or a substituted or unsubstituted heterocycle.

Preferred residues R¹, R² and R³ in the above-stated formula (C-III) are substituted or unsubstituted, straight-chain or branched alkyl residues. Among these, alkyl residues with 1 to 5 carbon atoms and hydroxyalkyl residues are preferred, such that preferred agents according to the invention are characterized in that the residues R¹, R² and R³ in the formula (C-III) are selected from methyl, ethyl, n-propyl, iso-propyl, n-butyl, iso-butyl, tert.-butyl as well as hydroxymethyl and hydroxyethyl residues.

Preferred residues R⁴ in the above-stated formula (C-III) are hydrogen, substituted or unsubstituted, straight-chain or branched alkyl residues as well as trialkylsilyl residues. Among these, hydrogen, methyl, ethyl, tert.-butyl and trimethylsilyl residues are preferred.

The acidic H atom of the silyl carbonate (R⁴═—H in formula C-III) may also assume neutralized form, i.e. salts of silyl carbonates may also be used according to the invention. Agents which are preferred according to the invention are here those which contain at least one silyl carbonate in entirely or partially neutralized form, preferably in the form of the alkali metal, ammonium, alkaline earth metal or aluminum salt thereof and in particular in the form of the sodium salt thereof.

In addition to or instead of the silyl carbonates, the agents according to the invention may contain silyl carbamates.

Agents which are preferred according to the invention are characterized in that they contain a silyl carbamate of the formula (C-IV)

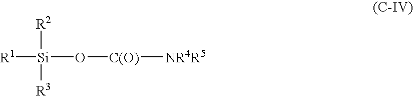

in which the residues R¹, R² and R³ mutually independently denote a hydrogen atom, a saturated or unsaturated, straight-chain, branched, or cyclic, substituted or unsubstituted hydrocarbon residue or a trialkylsilyl group, preferably a trimethylsilyl group or a substituted or unsubstituted aryl group or a substituted or unsubstituted heterocycle or a halogen, a substituted or unsubstituted hydroxy, oxo, amino group and the residues R⁴ and R⁵ mutually independently denote a chemical bond to the Si atom or to one of the residues R¹, R² or R³, a hydrogen atom, a saturated or unsaturated, straight-chain, branched, or cyclic, substituted or unsubstituted hydrocarbon residue or a substituted or unsubstituted silyl or aluminyl group or a substituted or unsubstituted aryl group or a substituted or unsubstituted heterocycle.

Preferred residues R¹, R² and R³ in the above-stated formula (C-IV) are substituted or unsubstituted, straight-chain or branched alkyl residues. Among these, alkyl residues with 1 to 5 carbon atoms and hydroxyalkyl residues are preferred, such that preferred agents according to the invention are characterized in that the residues R¹, R² and R³ in the formula (VII) are selected from methyl, ethyl, n-propyl, iso-propyl, n-butyl, iso-butyl, tert.-butyl as well as hydroxymethyl and hydroxyethyl residues.

Preferred residues R⁴ and R⁵ in the above-stated formula (C-IV) are hydrogen, substituted or unsubstituted, straight-chain or branched alkyl residues as well as trialkylsilyl residues. Among these, hydrogen, methyl, ethyl, tert.-butyl and trimethylsilyl residues are preferred.

Irrespective of whether the agents according to the invention contain one or more silyl carbonates and/or one or more silyl carbamates, agents according to the invention which are preferred are those which contain the silyl carbonate(s) and/or silyl carbamate(s) in quantities of 0.1 to 25 wt. %, preferably of 0.5 to 20 wt. %, particularly preferably of 1 to 15 wt. % and in particular of 1.5 to 10 wt. %, in each case relative to the entire agent.

A further increase in lightening power may be achieved by the addition of cyanates or “cyanate analogs”, which enable action to be enhanced. Agents which are preferred according to the invention may therefore contain 0.05 to 5 wt. % of at least one cyanate of the formula

M⁺[OCN]⁻
in which M⁺ denotes K⁺, Na⁺, Li⁺ or NH₄⁺ or ½Ca²⁺, ½Mg²⁺ or ½Zn²⁺
   and/or 0.05 to 5 wt % of at least one compound of the formula

H₂N—C(O)-L

in which L denotes a group displaceable by the anion ⁻OOH.

Cyanates which may be used according to the invention are potassium cyanate, KOCN, and/or sodium cyanate, NaOCN, and/or lithium cyanate, LiOCN, and/or ammonium cyanate, NH₄OCN, and/or calcium cyanate, Ca(OCN)₂, and/or magnesium cyanate, Mg(OCN)₂, and/or zinc cyanate, Zn(OCN)₂.

Agents which are preferred according to the invention are characterized in that they contain 0.075 to 4 wt. %, preferably 0.1 to 2.5 wt. % and in particular 0.2 to 1 wt. % of sodium cyanate and/or potassium cyanate and/or ammonium cyanate, sodium cyanate being particularly preferred.

Instead of cyanates or in combination therewith, compounds of the formula H₂N—C(O)-L, in which L denotes a group displaceable by the anion ⁻OOH, may also be present in the agents according to the invention. Agents which are preferred according to the invention are here characterized in that they contain 0.075 to 4 wt. %, preferably 0.1 to 3.5 wt. % and in particular 0.2 to 3 wt. % of at least one compound of the formula

H₂N—C(O)-L

in which L is selected from —S—CH₂—COOH, —S—(CH₂)₃SO₃H, —S—(CH₂)₂—NH₂, —OCH₃, —O—(C₆H₄)—SO₃M, -Q⁺, —NH—OH, —O—C(O)—R¹, —O—C(O)—H, —O—CH(OH)₂, —SO₃M, —PH(O)OH, —P(O)(OH)₂, —(NH-glucosamine)ₙ, —NH—C(O)—NH₂, —NH—NH₂, —NH—CN, —P(OH)(O)—C(O)NH₂, —SCN, —OCN, —S—C(NH)NH₂,

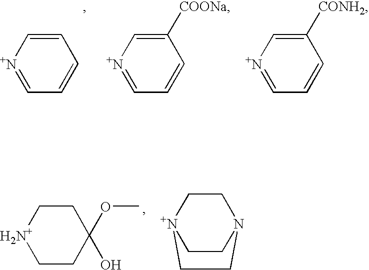

and M denotes H, Na, K, NH₄, Q is selected from quaternary ammonium residues formed from tertiary amines, heteroaryl residues or nonaromatic heterocycles and R¹ denotes —H, —CH₃, —CH₂CH₃, —(CH₂)₂CH₃, —CH(CH₃)₂ or another alkyl group.

Agents which are particularly preferred according to the invention contain 0.075 to 4 wt. %, preferably 0.1 to 3.5 wt. % and in particular 0.2 to 3 wt. % of at least one of the compounds listed below:
H₂N—C(O)—S—CH₂—COOH
H₂N—C(O)—S—(CH₂)₂—NH₂
H₂N—C(O)—S—(CH₂)₃SO₃Na
H₂N—C(O)—S—(CH₂)₃SO₃K
H₂N—C(O)—S—(CH₂)₃SO₃NH₄
H₂N—C(O)—SO₃H
H₂N—C(O)—SO₃Na
H₂N—C(O)—SO₃K
H₂N—C(O)—SO₃NH₄
H₂N—C(O)—O—(C₆H₄)—SO₃H
H₂N—C(O)—O—(C₆H₄)—SO₃Na
H₂N—C(O)—O—(C₆H₄)—SO₃K
H₂N—C(O)—O—(C₆H₄)—SO₃NH₄
H₂N—C(O)—O—C(O)—H
H₂N—C(O)—O—C(O)—CH₃
H₂N—C(O)—O—C(O)—CH₂CH₃
H₂N—C(O)—O—C(O)—(CH₂)₂CH₃
H₂N—C(O)—O—C(O)—CH(CH₃)₂
H₂N—C(O)—OCH₃
H₂N—C(O)—NH—OH
H₂N—C(O)—O—CH(OH)₂
H₂N—C(O)—PH(O)OH
H₂N—C(O)—P(O)(OH)₂
H₂N—C(O)—NH—C(O)—NH₂
H₂N—C(O)—NH—NH₂
H₂N—C(O)—NH—CN
H₂N—C(O)—P(OH)(O)—C(O)NH₂
H₂N—C(O)—SCN
H₂N—C(O)—OCN
H₂N—C(O)—S—C(NH)NH₂
H₂N—C(O)—N(CH₃)₃⁺Cl⁻
H₂N—C(O)—N(CH₂CH₃)₃⁺Cl⁻
H₂N—C(O)—N((CH₂)₂CH₃)₃⁺Cl⁻
H₂N—C(O)—N(CH(CH₃)₂)₃⁺Cl⁻
H₂N—C(O)—N(C₆H₅)₃⁺Cl⁻
H₂N—C(O)—N(CH₂-Ph)₃⁺Cl⁻
H₂N—C(O)—N(CH₃)₃⁺Br⁻
H₂N—C(O)—N(CH₂CH₃)₃⁺Br⁻
H₂N—C(O)—N((CH₂)₂CH₃)₃⁺Br⁻
H₂N—C(O)—N(CH(CH₃)₂)₃⁺Br⁻
H₂N—C(O)—N(C₆H₅)₃⁺Br⁻
H₂N—C(O)—N(CH₂-Ph)₃⁺Br⁻
H₂N—C(O)—N(CH₃)₃⁺H₃C—OSO₃⁻
H₂N—C(O)—N(CH₂CH₃)₃⁺H₃C—OSO₃⁻
H₂N—C(O)—N((CH₂)₂CH₃)₃⁺H₃C—OSO₃⁻
H₂N—C(O)—N(CH(CH₃)₂)₃⁺H₃C—OSO₃⁻
H₂N—C(O)—N(C₆H₅)₃⁺H₃C—OSO₃⁻
H₂N—C(O)—N(CH₂-Ph)₃⁺H₃C—OSO₃⁻
H₂N—C(O)—N(CH₃)₃⁺H₃C—CH₂—OSO₃⁻
H₂N—C(O)—N(CH₂CH₃)₃⁺H₃C—CH₂—OSO₃⁻
H₂N—C(O)—N((CH₂)₂CH₃)₃⁺H₃C—CH₂—OSO₃⁻
H₂N—C(O)—N(CH(CH₃)₂)₃⁺H₃C—CH₂—OSO₃⁻
H₂N—C(O)—N(C₆H₅)₃⁺H₃C—CH₂—OSO₃⁻
H₂N—C(O)—N(CH₂-Ph)₃⁺H₃C—CH₂—OSO₃⁻

Piperidones or the derivatives thereof may also enhance the effects, such that it is preferred to use 0.1 to 20 wt. % of at least one piperidone and/or piperidone derivative in the agents according to the invention. The agents according to the invention preferably contain 0.5 to 15 wt. %, particularly preferably 1 to 12.5 wt. % and in particular 2.5 to 10 wt. % of at least one compound from the group
2-piperidone and/or
derivatives of 2-piperidone, in particular 2-piperidinium salts and/or
3-piperidone and/or
derivatives of 3-piperidone, in particular 3-piperidinium salts and/or
4-piperidone and/or
derivatives of 4-piperidone, in particular 4-piperidinium salts.

Piperidones are compounds which comprise a keto function on a six-membered ring which is formed from five carbon atoms and a nitrogen atom. Depending on the position of the nitrogen atom relative to the keto group, the unsubstituted parent substance is designated 2-piperidone (piperidin-2-one, δ-valerolactam, alpha-piperidone), 3-piperidone (piperidin-3-one, beta-piperidone) or 4-piperidone (piperidin-4-one, gamma-piperidone).

These three piperidones are preferably usable according to the invention. Piperidone derivatives which may be considered for use according to the invention in addition to or instead of the piperidones are both those piperidones substituted on the C skeleton and those substituted on the nitrogen atom, the N-substituted piperidone derivatives being of great significance, since the H atom on the nitrogen can readily be replaced. Among these piperidone derivatives, the following have proved to be particularly preferred according to the invention:
1-methyl-piperidin-4-one (N-methyl-4-piperidone)
1-methyl-piperidin-3-one (N-methyl-3-piperidone)
1-methyl-piperidin-2-one (N-methyl-2-piperidone)
1-ethyl-piperidin-4-one (N-ethyl-4-piperidone)
1-ethyl-piperidin-3-one (N-ethyl-3-piperidone)
1-ethyl-piperidin-2-one (N-ethyl-2-piperidone)
1-propyl-piperidin-4-one (N-propyl-4-piperidone)
1-propyl-piperidin-3-one (N-propyl-3-piperidone)
1-propyl-piperidin-2-one (N-propyl-2-piperidone)
1-isopropyl-piperidin-4-one (N-isopropyl-4-piperidone)
1-isopropyl-piperidin-3-one (N-isopropyl-3-piperidone)
1-isopropyl-piperidin-2-one (N-isopropyl-2-piperidone)
1-butyl-piperidin-4-one (N-butyl-4-piperidone)
1-butyl-piperidin-3-one (N-butyl-3-piperidone)
1-butyl-piperidin-2-one (N-butyl-2-piperidone)
1-pentyl-piperidin-4-one (N-pentyl-4-piperidone)
1-pentyl-piperidin-3-one (N-pentyl-3-piperidone)
1-pentyl-piperidin-2-one (N-pentyl-2-piperidone)
1-hexyl-piperidin-4-one (N-hexyl-4-piperidone)
1-hexyl-piperidin-3-one (N-hexyl-3-piperidone)
1-hexyl-piperidin-2-one (N-hexyl-2-piperidone)
1-heptyl-piperidin-4-one (N-heptyl-4-piperidone)
1-heptyl-piperidin-3-one (N-heptyl-3-piperidone)
1-heptyl-piperidin-2-one (N-heptyl-2-piperidone)
1-optyl-piperidin-4-one (N-octyl-4-piperidone)
1-optyl-piperidin-3-one (N-octyl-3-piperidone)
1-octyl-piperidin-2-one (N-octyl-2-piperidone)
1-nonyl-piperidin-4-one (N-nonyl-4-piperidone)
1-nonyl-piperidin-3-one (N-nonyl-3-piperidone)
1-nonyl-piperidin-2-one (N-nonyl-2-piperidone)
1-decyl-piperidin-4-one (N-decyl-4-piperidone)
1-decyl-piperidin-3-one (N-decyl-3-piperidone)
1-decyl-piperidin-2-one (N-decyl-2-piperidone)

The N atom in the piperidones preferably used according to the invention may also assume quaternized form, such that piperidinium salts may preferably also be used according to the invention. Agents which are particularly preferred according to the invention are here characterized in that they contain 3-piperidinium salts of the formula (IV) and/or 4-piperidinium salts of the formula (V)

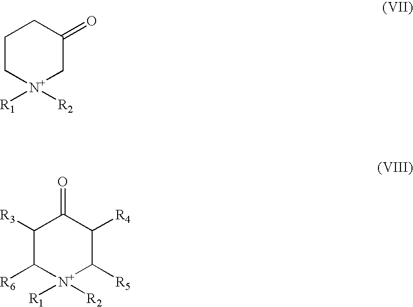

in which the residues R¹ or R² or R³ or R⁴ or R⁵ or R⁶ are mutually independently selected from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, or other alkyl residues, alkenyl residues, optionally substituted aryl residues and in which the counterion is preferably selected from chloride, bromide, methosulfate, toluenesulfonate.

The residues R³ or R⁴ or R⁵ or R⁶ may preferably be a hydrogen atom. Among these piperidinium salts, the following have proved to be particularly preferred according to the invention:
3-oxo-piperidinium chloride (R¹═R²═H in formula VII)
4-oxo-piperidinium chloride (R¹═R²═H in formula VIII)
3-oxo-piperidinium bromide (R¹═R²═H in formula VII)
4-oxo-piperidinium bromide (R¹═R²═H in formula VIII)
3-oxo-piperidinium methosulfate (R¹═R²═H in formula VII)
4-oxo-piperidinium methosulfate (R¹═R²═H in formula VIII)
3-oxo-piperidinium tosylate (R¹═R²═H in formula VII)
4-oxo-piperidinium tosylate (R¹═R²═H in formula VIII)
N-methyl-3-oxopiperidinium chloride (R¹═—CH₃, R²═H in formula VII)
N-methyl-4-oxopiperidinium chloride (R¹═—CH₃, R²═H in formula VIII)
N-methyl-3-oxopiperidinium bromide (R¹═—CH₃, R²═H in formula VII)
N-methyl-4-oxopiperidinium bromide (R¹═—CH₃, R²═H in formula VIII)
N-methyl-3-oxopiperidinium methosulfate (R¹═—CH₃, R²═H in formula VII)
N-methyl-4-oxopiperidinium methosulfate (R¹═—CH₃, R²═H in formula VIII)
N-methyl-3-oxopiperidinium tosylate (R¹═—CH₃, R²═H in formula VII)
N-methyl-4-oxopiperidinium tosylate (R¹═—CH₃, R²═H in formula VIII)
N-ethyl-3-oxopiperidinium chloride (R¹═—CH₂CH₃, R²═H in formula VII)
N-ethyl-4-oxopiperidinium chloride (R¹═—CH₂CH₃, R²═H in formula VIII)
N-ethyl-3-oxopiperidinium bromide (R¹═—CH₂CH₃, R²═H in formula VII)
N-ethyl-4-oxopiperidinium bromide (R¹═—CH₂CH₃, R²═H in formula VIII)
N-ethyl-3-oxopiperidinium methosulfate (R¹═—CH₂CH₃, R²═H in formula VII)
N-ethyl-4-oxopiperidinium methosulfate (R¹═—CH₂CH₃, R²═H in formula VIII)
N-ethyl-3-oxopiperidinium tosylate (R¹═—CH₂CH₃, R²═H in formula VII)
N-ethyl-4-oxopiperidinium tosylate (R¹═—CH₂CH₃, R²═H in formula VIII)
N-propyl-3-oxopiperidinium chloride (R¹═—CH₂CH₂CH₃, R²═H in formula VII)
N-propyl-4-oxopiperidinium chloride (R¹═—CH₂CH₂CH₃, R²═H in formula VIII)
N-propyl-3-oxopiperidinium bromide (R¹═—CH₂CH₂CH₃, R²═H in formula VII)
N-propyl-4-oxopiperidinium bromide (R¹═—CH₂CH₂CH₃, R²═H in formula VIII)
N-propyl-3-oxopiperidinium methosulfate (R¹═—CH₂CH₂CH₃, R²═H in formula VII)
N-propyl-4-oxopiperidinium methosulfate (R¹═—CH₂CH₂CH₃, R²═H in formula VIII)
N-propyl-3-oxopiperidinium tosylate (R¹═—CH₂CH₂CH₃, R²═H in formula VII)
N-propyl-4-oxopiperidinium tosylate (R¹═—CH₂CH₂CH₃, R²═H in formula VIII)
N-isopropyl-3-oxopiperidinium chloride (R¹═—CH(CH₃)₂, R²═H in formula VII)
N-isopropyl-4-oxopiperidinium chloride (R¹═—CH(CH₃)₂, R²═H in formula VIII)
N-isopropyl-3-oxopiperidinium bromide (R¹═—CH(CH₃)₂R²═H in formula VII)
N-isopropyl-4-oxopiperidinium bromide (R¹═—CH(CH₃)₂, R²═H in formula VIII)
N-isopropyl-3-oxopiperidinium methosulfate (R¹═—CH(CH₃)₂, R²═H in formula VII)
N-Isopropyl-4-oxopiperidinium methosulfate (R¹═—CH(CH₃)₂, R²═H in formula VIII)
N-isopropyl-3-oxopiperidinium tosylate (R¹═—CH(CH₃)₂, R²═H in formula VII)
N-isopropyl-4-oxopiperidinium tosylate (R¹═—CH(CH₃)₂, R²═H in formula VIII)
N-butyl-3-oxopiperidinium chloride (R¹═—(CH₂)₃CH₃, R²═H in formula VII)
N-butyl-4-oxopiperidinium chloride (R¹═—(CH₂)₃CH₃, R²═H in formula VIII)
N-butyl-3-oxopiperidinium bromide (R¹═—(CH₂)₃CH₃, R²═H in formula VII)
N-butyl-4-oxopiperidinium bromide (R¹═—(CH₂)₃CH₃, R²═H in formula VIII)
N-butyl-3-oxopiperidinium methosulfate (R¹═—(CH₂)₃CH₃, R²═H in formula VII)
N-butyl-4-oxopiperidinium methosulfate (R¹═—(CH₂)₃CH₃, R²═H in formula VIII)
N-butyl-3-oxopiperidinium tosylate (R¹═—(CH₂)₃CH₃, R²═H in formula VII)
N-butyl-4-oxopiperidinium tosylate (R¹═—(CH₂)₃CH₃, R²═H in formula VIII)
N-isobutyl-3-oxopiperidinium chloride (R¹═—CH₂CH(CH₃)₂, R²═H in formula VII)
N-isobutyl-4-oxopiperidinium chloride (R¹═—CH₂CH(CH₃)₂, R²═H in formula VIII)
N-Isobutyl-3-oxopiperidinium bromide (R¹═—CH₂CH(CH₃)₂, R²═H in formula VII)
N-isobutyl-4-oxopiperidinium bromide (R¹═—CH₂CH(CH₃)₂, R²═H in formula VIII)
N-isobutyl-3-oxopiperidinium methosulfate (R¹═—CH₂CH(CH₃)₂, R²═H in formula VII)
N-isobutyl-4-oxopiperidinium methosulfate (R¹═—CH₂CH(CH₃)₂, R²═H in formula VIII)
N-isobutyl-3-oxopiperidinium tosylate (R¹═—CH₂CH(CH₃)₂, R²═H in formula VII)
N-isobutyl-4-oxopiperidinium tosylate (R¹═—CH₂CH(CH₃)₂, R²═H in formula VIII)
N-sec-butyl-3-oxopiperidinium chloride (R¹═—CH₂CH(CH₃)₂, R²═H in formula VII)
N-sec-butyl-4-oxopiperidinium chloride (R¹═—CH₂CH(CH₃)₂, R²═H in formula VIII)
N-sec-butyl-3-oxopiperidinium bromide (R¹═—CH₂CH(CH₃)₂, R²═H in formula VII)
N-sec-butyl-4-oxopiperidinium bromide (R¹═—CH₂CH(CH₃)₂, R²═H in formula VIII)
N-sec-butyl-3-oxopiperidinium methosulfate (R¹═—CH₂CH(CH₃)₂, R²═H in formula VII)
N-sec-butyl-4-oxopiperidinium methosulfate (R¹═—CH₂CH(CH₃)₂, R²═H in formula VIII)
N-sec-butyl-3-oxopiperidinium tosylate (R¹═—CH₂CH(CH₃)₂, R²═H in formula VII)
N-sec-butyl-4-oxopiperidinium tosylate (R¹═—CH₂CH(CH₃)₂, R²═H in formula VIII)
N,N-dimethyl-3-oxopiperidinium chloride (R¹═R²═—CH₃ in formula VII)
N,N-dimethyl-4-oxopiperidinium chloride (R¹═R²′—CH₃ in formula VIII)
N,N-dimethyl-3-oxopiperidinium bromide (R¹═R²′—CH₃ in formula VII)
N,N-dimethyl-4-oxopiperidinium bromide (R¹═R²═—CH₃ in formula VIII)
N,N-dimethyl-3-oxopiperidinium methosulfate (R¹═R²═—CH₃ in formula VII)
N,N-dimethyl-4-oxopiperidinium methosulfate (R¹═R²═—CH₃ in formula VIII)
N,N-dimethyl-3-oxopiperidinium tosylate (R¹═R²═—CH₃ in formula VII)
N,N-dimethyl-4-oxopiperidinium tosylate (R¹═R²═—CH₃ in formula VIII)
N,N-diethyl-3-oxopiperidinium chloride (R¹═—CH₂CH₃, R²═H in formula VII)
N,N-diethyl-4-oxopiperidinium chloride (R¹═—CH₂CH₃, R²═H in formula VIII)
N,N-diethyl-3-oxopiperidinium bromide (R¹═—CH₂CH₃, R²═H in formula VII)

N,N-diethyl-4-oxopiperidinium bromide (R¹═—CH₂CH₃, R²═H in formula VIII)
N,N-diethyl-3-oxopiperidinium methosulfate (R¹═—CH₂CH₃, R²═H in formula VII)
N,N-diethyl-4-oxopiperidinium methosulfate (R¹═—CH₂CH₃, R²═H in formula VIII)
N,N-diethyl-3-oxopiperidinium tosylate (R¹═—CH₂CH₃, R²═H in formula VII)
N,N-diethyl-4-oxopiperidinium tosylate (R¹═—CH₂CH₃, R²═H in formula VIII)
N,N-dipropyl-3-oxopiperidinium chloride (R¹═R²═—CH₂CH₂CH₃ in formula VII)
N,N-dipropyl-4-oxopiperidinium chloride (R¹═R²═—CH₂CH₂CH₃ in formula VIII)
N,N-dipropyl-3-oxopiperidinium bromide (R¹═R²═—CH₂CH₂CH₃ in formula VII)
N,N-dipropyl-4-oxopiperidinium bromide (R¹═R²═—CH₂CH₂CH₃ in formula VIII)
N,N-dipropyl-3-oxopiperidinium methosulfate (R¹═R²═—CH₂CH₂CH₃ in formula VII)
N,N-dipropyl-4-oxopiperidinium methosulfate (R¹═R²′—CH₂CH₂CH₃ in formula VIII)
N,N-dipropyl-3-oxopiperidinium tosylate (R¹═R²═—CH₂CH₂CH₃ in formula VII)
N,N-dipropyl-4-oxopiperidinium tosylate (R¹═R²═—CH₂CH₂CH₃ in formula VIII)
N,N-diisopropyl-3-oxopiperidinium chloride (R¹═R²═—CH(CH₃)₂ in formula VII)
N,N-diisopropyl-4-oxopiperidinium chloride (R¹═R²═—CH(CH₃)₂ in formula VIII)
N,N-diisopropyl-3-oxopiperidinium bromide (R¹═R²═—CH(CH₃)₂ in formula VII)
N,N-diisopropyl-4-oxopiperidinium bromide (R¹═R²═—CH(CH₃)₂ in formula VIII)
N,N-diisopropyl-3-oxopiperidinium methosulfate (R¹═R²═—CH(CH₃)₂ in formula VII)
N,N-diisopropyl-4-oxopiperidinium methosulfate (R¹═R²═—CH(CH₃)₂ in formula VIII)
N,N-diisopropyl-3-oxopiperidinium tosylate (R¹═R²═—CH(CH₃)₂ in formula VII)
N,N-diisopropyl-4-oxopiperidinium tosylate (R¹═R²═—CH(CH₃)₂ in formula VIII)
N,N-dibutyl-3-oxopiperidinium chloride (R¹═R²═—(CH₂)₃CH₃ in formula VII)
N,N-dibutyl-4-oxopiperidinium chloride (R¹═R²═—(CH₂)₃CH₃ in formula VIII)
N,N-dibutyl-3-oxopiperidinium bromide (R¹═R²═—(CH₂)₃CH₃ in formula VII)
N,N-dibutyl-4-oxopiperidinium bromide (R¹═R²═—(CH₂)₃CH₃ in formula VIII)
N,N-dibutyl-3-oxopiperidinium methosulfate (R¹═R²═—(CH₂)₃CH₃ in formula VII)
N,N-dibutyl-4-oxopiperidinium methosulfate (R¹═R²═—(CH₂)₃CH₃ in formula VIII)
N,N-dibutyl-3-oxopiperidinium tosylate (R¹═R²═—(CH₂)₃CH₃ in formula VII)
N,N-dibutyl-4-oxopiperidinium tosylate (R¹═R²═—(CH₂)₃CH₃ in formula VIII)
N,N-diisobutyl-3-oxopiperidinium chloride (R¹═—CH₂CH(CH₃)₂, R²═H in formula VII)
N,N-diisobutyl-4-oxopiperidinium chloride (R¹═—CH₂CH(CH₃)₂, R²═H in formula VIII)
N,N-diisobutyl-3-oxopiperidinium bromide (R¹═—CH₂CH(CH₃)₂, R²═H in formula VII)
N,N-diisobutyl-4-oxopiperidinium bromide (R¹═—CH₂CH(CH₃)₂, R²═H in formula VIII)
N,N-diisobutyl-3-oxopiperidinium methosulfate (R¹═—CH₂CH(CH₃)₂, R²═H in formula VII)
N,N-diisobutyl-4-oxopiperidinium methosulfate (R¹═—CH₂CH(CH₃)₂, R²═H in formula VIII)
N,N-diisobutyl-3-oxopiperidinium tosylate (R¹═—CH₂CH(CH₃)₂, R²═H in formula VII)
N,N-diisobutyl-4-oxopiperidinium tosylate (R¹═—CH₂CH(CH₃)₂, R²═H in formula VIII)
N,N-di(sec-butyl)-3-oxopiperidinium chloride (R¹═R²═—CH₂CH(CH₃)₂ in formula VII)
N,N-di(sec-butyl)-4-oxopiperidinium chloride (R¹═R²═—CH₂CH(CH₃)₂ in formula VIII)
N,N-di(sec-butyl)-3-oxopiperidinium bromide (R¹═R²═—CH₂CH(CH₃)₂ in formula VII)
N,N-di(sec-butyl)-4-oxopiperidinium bromide (R¹═R²═—CH₂CH(CH₃)₂ in formula VIII))
N,N-di(sec-butyl)-3-oxopiperidinium methosulfate (R¹═R²═—CH₂CH(CH₃)₂ in formula VII)
N,N-di(sec-butyl)-4-oxopiperidinium methosulfate (R¹═R²═—CH₂CH(CH₃)₂ in formula VIII)
N,N-di(sec-butyl)-3-oxopiperidinium tosylate (R¹═R²═—CH₂CH(CH₃)₂ in formula VII))
N,N-di(sec-butyl)-4-oxopiperidinium tosylate (R¹═R²═—CH₂CH(CH₃)₂ in formula VIII)
N,N-3,5-tetramethyl-4-oxopiperidinium iodide (R¹═R²═R³═R⁴═—CH₃ in formula VIII)
N,N-2,6-tetramethyl-4-oxopiperidinium iodide (R¹═R²═R⁶═R⁶═—CH₃ in formula VIII)

It has furthermore proven advantageous for the dyes and/or lightening agents according to the invention to contain nonionogenic interfacially active substances.

Such interfacially active substances which have an HLB value of 5.0 and above are here preferred. With regard to the definition of HLB value, explicit reference is made to the explanations in Hugo Janistyn, Handbuch der Kosmetika und Riechstoffe, volume III: Die Körperpflegemittel, 2nd edition, Dr. Alfred Hüthig Verlag Heidelberg, 1973, pages 68-78 and Hugo Janistyn, Taschenbuch der modernen Parfümerie und Kosmetik, 4th edition, Wissenschaftliche Verlagsgesellschaft m.b.H. Stuttgart, 1974, pages 466-474 as well as the original papers cited therein.

Particularly preferred nonionogenic surface-active substances are here, due to ease of processing, substances which are commercially available in pure form as solids or liquids. The definition of purity here does not refer to chemically pure compounds. Instead, in particular in relation to naturally based products, mixtures of different homologs may be used, for example with different alkyl chain lengths, as are obtained in the case of products based on natural fats and oils. In the case of alkoxylated products too, mixtures of differing degrees of alkoxylation are conventionally present. The term purity instead refers in this connection to the fact that the selected substances should preferably contain no solvents, adjusting agents and other accompanying substances.

Preferred nonionogenic interfacially active substances are
alkoxylated fatty alcohols with 8 to 22, in particular 10 to 16, carbon atoms in the fatty alkyl group and 1 to 30, in particular 1 to 15, ethylene oxide and/or propylene oxide units. Preferred fatty alkyl groups are for example not only lauryl, myristyl, cetyl, but also stearyl, isostearyl and oleyl groups. Particularly preferred compounds of this class are for example lauryl alcohol with 2 to 4 ethylene oxide units, oleyl and cetyl alcohol with in each case 5 to 10 ethylene oxide units, cetyl and stearyl alcohol as well as mixtures thereof with 10 to 30 ethylene oxide units as well as the commercial product Aethoxal®B (Henkel), a lauryl alcohol with in each case 5 ethylene oxide and propylene oxide units. In addition to conventional alkoxylated fatty alcohols, “end group-terminated” compounds may also be used according to the invention. In these compounds, the alkoxy group does not have a terminal OH group, but is instead “terminated” in the form of an ether, in particular a C₁-C₄ alkyl ether. One example of such a compound is the commercial product Dehypon®LT 054, a C₁₂₋₁₈ fatty alcohol+4.5 ethylene oxide butyl ether.
alkoxylated fatty acids with 8 to 22, in particular 10 to 16, carbon atoms in the fatty acid group and 1 to 30, in particular 1 to 15, ethylene oxide and/or propylene oxide units. Preferred fatty acids are for example lauric, myristic, palmitic, stearic, isostearic and oleic acid.
alkoxylated, preferably propoxylated and in particular ethoxylated, mono-, di- and triglycerides. Examples of preferred compounds are glycerol monolaurate+20 ethylene oxide and glycerol monostearate+20 ethylene oxide.
polyglycerol esters and alkoxylated polyglycerol esters. Preferred compounds of this class are for example poly(3)glycerol diisostearate (commercial product: Lameform®TGI (Henkel)) and poly(2)glycerol polyhydroxystearate (commercial product: Dehymuls®PGPH (Henkel)).
sorbitan fatty acid esters and alkoxylated sorbitan fatty acid esters such as for example sorbitan monolaurate and sorbitan monolaurate+20 ethylene oxide (EO).
alkylphenols and alkylphenol alkoxylates with 6 to 21, in particular 6 to 15, carbon atoms in the alkyl chain and 0 to 30 ethylene oxide and/or propylene oxide units. Preferred representatives of this class are for example nonylphenol+4 EO, nonylphenol+9 EO, octylphenol+3 EO and octylphenol+8 EO.

The alkoxylated fatty alcohols, alkoxylated fatty acids as well as alkylphenols and alkylphenol alkoxylates are particularly preferred classes of nonionogenic interfacially active substances.

Agents according to the invention which contain the nonionogenic interfacially active substances in quantities of 1-5 wt. % have proved particularly advantageous.

The dyes and/or lightening agents according to the invention may furthermore contain any active ingredients, additives and auxiliary substances known in such preparations. In many cases, the agents contain at least one surfactant, with not only anionic but also in principle zwitterionic, ampholytic, nonionic and cationic surfactants being suitable. In many cases, however, it has proven advantageous to select the surfactants from among anionic, cationic or nonionic surfactants. Anionic surfactants may here be very particularly preferred.

Preferred anionic surfactants are alkyl sulfates, ether carboxylic acid salts with 10 to 18 C atoms in the alkyl group and up to 12 glycol ether groups per molecule such as C₁₂H₂₅—(C₂H₄O)₆—CH₂—COONa as well as in particular salts of saturated and specifically unsaturated C₈-C₂₂ carboxylic acids such as oleic acid, stearic acid, isostearic acid and palmitic acid.

These anionic surfactants should preferably assume solid, in particular powder, form. Soaps which are solid at room temperature, in particular sodium stearate, are very particularly preferred. These are preferably present in quantities of 5 to 20 wt. %, in particular of 10 to 15 wt. %.

Suitable nonionic surfactants are in particular C8-C22 alkyl mono- and oligoglycosides and the ethoxylated analogs thereof. Nonethoxylated compounds have in particular proved particularly suitable.

Examples of cationic surfactants usable in the hair treatment agents according to the invention are in particular quaternary ammonium compounds. Ammonium halides are preferred, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Quaternized protein hydrolysates are further cationic surfactants which are usable according to the invention.

Alkylamidoamines, in particular fatty acid amidoamines such as stearylamidopropyldimethylamine obtainable under the name Tego Amide®S 18 are distinguished by good biodegradability in addition having a good conditioning action.

Quaternary ester compounds, known as “ester quats”, such as the distearoylethylhydroxyethylammonium methosulfate blended with cetearyl alcohol obtainable under the name Dehyquart®F 75 are likewise very highly biodegradable.

The compounds with alkyl groups used as surfactants may in each case comprise uniform substances. It is, however, generally preferred to start from native plant or animal raw materials when producing these substances, such that mixtures of substances having differing alkyl chain lengths depending on the particular raw material are obtained.

The compositions according to the invention may contain at least one ammonium compound from the group ammonium chloride, ammonium carbonate, ammonium bicarbonate, ammonium sulfate and/or ammonium carbamate in a quantity of 0.5 to 10, preferably 1 to 5 wt. %, relative to the total composition of the agent, as a further constituent.

The dyes and/or lightening agents according to the invention may moreover contain further active ingredients, auxiliary substances and additives, such as for example
nonionic polymers such as for example vinylpyrrolidone/vinyl acrylate copolymers,
polyvinylpyrrolidone and vinylpyrrolidone/vinyl acetate copolymers and polysiloxanes,
cationic polymers such as quaternized cellulose ethers, polysiloxanes with quaternary groups, dimethyldiallylammonium chloride polymers, acrylamide/dimethyldiallylammonium chloride copolymers, dimethyl-aminoethyl methacrylate/vinylpyrrolidone copolymers quaternized with diethyl sulfate, vinylpyrrolidone/imidazolinium methochloride copolymers and quaternized polyvinyl alcohol,
zwitterionic and amphoteric polymers such as for example acrylamidopropyltrimethylammonium chloride/acrylate copolymers, and octylacrylamide/methyl methacrylate/tert-butylaminoethyl methacrylate/2-hydroxypropyl methacrylate copolymers,
anionic polymers such as for example polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butylacrylamide terpolymers,
thickeners such as agar-agar, guar gum, alginates, xanthan gum, gum arabic, karaya gum, locust bean flour, linseed gums, dextrans, cellulose derivatives, for example methylcellulose, hydroxyalkylcellulose and carboxymethylcellulose, starch fractions and derivatives such as amylase, amylopectin and dextrins, clays such as for example bentonite or completely synthetic hydrocolloids such as for example polyvinyl alcohol,
texturing agents such as maleic acid and lactic acid,
hair-conditioning compounds such as phospholipids, for example soy lecithin, egg lecithin and cephalins,
protein hydrolysates, in particular elastin, collagen, keratin, milk protein, soy protein and wheat protein hydrolysates, the condensation products thereof with fatty acids as well as quaternized protein hydrolysates,
perfume oils, dimethyl isosorbide and cyclodextrins,
solvents and solubilizing agents such as ethanol, isopropanol, ethylene glycol, propylene glycol, glycerol and diethylene glycol,
active ingredients which improve fiber structure, in particular mono-, di- and oligosaccharides such as for example glucose, galactose, fructose, fruit sugars and lactose,
quaternized amines such as methyl-1-alkylamidoethyl-2-alkylimidazolinium methosulfate
defoamers such as silicones,
dyes for coloring the preparation,
antidandruff active ingredients such as piroctone olamine, zinc omadine and climbazole,
light stabilizers, in particular derivatized benzophenones, cinnamic acid derivatives and triazines,
substances for adjusting pH value, such as for example conventional acids, in particular edible acids and bases,
active ingredients such as allantoin, pyrrolidonecarboxylic acids and the salts thereof as well as bisabolol,
vitamins, provitamins and vitamin precursors, in particular those of groups A, B₃, B₆, B₆, C, E, F and H,
plant extracts such as extracts from green tea, oak bark, stinging nettle, witch hazel, hops, chamomile, burdock root, horsetail, hawthorn, lime blossom, almond, aloe vera, pine-needle, horse chestnut, sandalwood, juniper, coconut, mango, apricot, lime, wheat, kiwi fruit, melon, orange, grapefruit, sage, rosemary, birch, mallow, lady's smock, wild thyme, yarrow, thyme, melissa, restharrow, coltsfoot, marsh mallow, meristem, ginseng and ginger root,
cholesterol,
consistency providers, such as sugar esters, polyol esters or polyol alkyl ethers,
fats and waxes such as spermaceti, beeswax, montan wax and paraffins,
fatty acid alkanolamides,
complexing agents such as EDTA, NTA, β-alaninediacetic acid and phosphonic acids,
swelling and penetrating substances such as glycerol, propylene glycol monoethyl ether, carbonates, hydrogencarbonates, guanidines, ureas and primary, secondary as well as tertiary phosphates,
opacifiers such as latex, styrene/PVP and styrene/acrylamide copolymers
pearlescent agents such as ethylene glycol mono- and distearate as well as PEG-3 distearate,
pigments,
stabilizers for hydrogen peroxide and other oxidizing agents,
propellants such as propane/butane mixtures, N₂O, dimethyl ether, CO₂ and air,
antioxidants.

With regard to further optional components and the quantities of these components used, reference is explicitly made to the relevant handbooks known to a person skilled in the art, for example Kh. Schrader, Grundlagen and Rezepturen der Kosmetika, 2nd edition, Huthig Buch Verlag, Heidelberg, 1989.

The agents according to the invention may contain the ingredients in a suitable aqueous, alcoholic or aqueous-alcoholic carrier. Carriers suitable for the purpose of hair dyeing are for example creams, emulsions, gels or also surfactant-containing foaming solutions, such as for example shampoos, foam aerosols or other preparations which are suitable for use on the hair. It is, however, also possible to provide a pulverulent or also tablet-shaped formulation, which is preferred for dyes and/or lightening agents.

For the purposes of the present invention, aqueous-alcoholic solutions should be taken to be aqueous solutions containing 3 to 70 wt. % of a C₁-C₄ alcohol, in particular ethanol or isopropanol. The agents according to the invention may additionally contain further organic solvents, such as for example methoxybutanol, benzyl alcohol, diethylene glycol monoethyl ether or 1,2-propylene glycol. Any water-soluble organic solvents are here preferred.

Preferred agents according to the invention are characterized in that they additionally contain a nonaqueous solvent, particularly preferred agents according to the invention containing the solvent in a concentration of 0.1-30 weight percent, preferably in a concentration of 1-20 weight percent, very particularly preferably in a concentration of 2-10 weight percent, in each case relative to the agent.

In further preferred agents according to the invention, the solvent is selected from ethanol, n-propanol, isopropanol, n-butanol, propylene glycol, n-butylene glycol, glycerol, diethylene glycol monoethyl ether, diethylene glycol mono-n-butyl ether, phenoxyethanol and benzyl alcohol as well as the mixtures thereof.

The pH value of the agents according to the invention may be adjusted within a wide range by suitable ingredients such as acidifying agents or alkalizing agents.

Oxidative dyeing of the fibers in the presence of oxidation dye precursors may in principle proceed with atmospheric oxygen. Preferably, however, a chemical oxidizing agent is used, particularly when it is wished to lighten human hair as well as dye it. This lightening effect may be desired independently of the dyeing method. The presence of oxidation dye precursors is accordingly not an essential prerequisite for using oxidizing agents in the agents according to the invention. Oxidizing agents which may be considered are persulfates, chlorites and in particular hydrogen peroxide or the addition products thereof onto urea, melamine as well as sodium borate.

According to the invention, however, the oxidation colorant may also be applied onto the hair together with a catalyst which activates the oxidation of the dye precursors, for example by atmospheric oxygen. Such catalysts are for example metal ions, iodides, quinones or certain enzymes.

Suitable metal ions are for example Zn²⁺, Cu²⁺, Fe²⁺, Fe³⁺, Mn²⁺, Mn⁴⁺, Li⁺, Mg²⁺, Ca²⁺ and Al³⁺. Zn²⁺, Cu²⁺ and Mn²⁺ are particularly suitable. The metal ions may in principle be used in the form of any desired, physiologically acceptable salt or in the form of a complex compound. Preferred salts are acetates, sulfates, halides, lactates and tartrates. By using these metal salts, it is possible both to accelerate dye formation and to have a targeted influence on color shade.

Suitable enzymes are for example peroxidases, which are capable of distinctly enhancing the action of small quantities of hydrogen peroxide. Enzymes which are furthermore suitable according to the invention are those which, with the assistance of atmospheric oxygen, directly oxidize the oxidation dye precursors, such as laccases for example, or which produce small quantities of hydrogen peroxide in situ and so biocatalytically activate oxidation of the dye precursors. Particularly suitable catalysts for the oxidation of dye precursors are “two-electron oxidoreductases” in combination with their specific substrates, for example
pyranose oxidase and for example D-glucose or galactose,
glucose oxidase and D-glucose,
glycerol oxidase and glycerol,
pyruvate oxidase and pyruvic acid or the salts thereof,
alcohol oxidase and alcohol (MeOH, EtOH),
lactate oxidase and lactic acid and the salts thereof,
tyrosinase oxidase and tyrosine,
uricase and uric acid or the salts thereof,
choline oxidase and choline,
amino acid oxidase and amino acids.

When oxidizing agents are used, the actual dye is conveniently produced immediately before use by mixing the oxidizing agent preparation with the preparation containing the compounds of the formula I and optionally dye precursors. The resultant ready-to-use hair dye preparation should preferably have a pH value in the range from 6 to 12. It is particularly preferable to apply the hair dye in a weakly alkaline medium. The application temperatures may lie in a range between 15 and 40° C. After an exposure time of 5 to 45 minutes, the hair dye is rinsed out of the hair to be dyed. Rewashing with a shampoo is not required if a carrier with an elevated surfactant content, e.g. a coloring shampoo, has been used.

In the case in particular of hair which is difficult to dye, however, an agent according to the invention optionally comprising additional dye precursors may also be applied onto the hair without prior mixing with the oxidation component. After an exposure time of 20 to 30 minutes, the oxidation component is then applied, optionally after intermediate rinsing. After a further exposure time of 10 to 20 minutes, the hair is then rinsed and reshampooed if desired. According to a first variant of this embodiment, in which prior application of the dye precursors is intended to bring about better penetration into the hair, the corresponding agent is adjusted to a pH value of approx. 4 to 7. According to a second variant, atmospheric oxidation is initially sought, the applied agent preferably having a pH value of 7 to 10. For the subsequent accelerated post-oxidation, the use of acidic peroxydisulfate solutions as oxidizing agent may be preferred.

For application of the agents according to the invention on the hair, the dyes and/or lightening agents are mixed with a hydrogen peroxide solution immediately prior to application. The concentration of this hydrogen peroxide solution is determined on the one hand by statutory requirements and on the other hand by the desired effect; as a rule, 6 to 12 percent solutions in water are used. The quantity ratios of dye and/or lightening agent and hydrogen peroxide solution are here conventionally in the range 1:1 to 1:2, an excess of hydrogen peroxide solution in particular being selected if a not excessively pronounced blonding action is desired.

The agents according to the invention may additionally contain further ingredients. It may, for example, be preferred to use specific metal ions or complexes in order to obtain intense colors. Agents according to the invention which additionally contain Cu, Fe, Mn, Ru ions or complexes of these ions are preferred for this purpose.

Preferred agents according to the invention for dyeing and/or lightening keratin fibers additionally contain Cu, Fe, Mn, Co, Ce, V, Ru ions or complexes of these ions, preferred agents [containing] 0.0001 to 2.5 wt. %, preferably 0.001 to 1 wt. % of at least one compound from the group copper chloride (CuCl₂), copper sulfate (CuSO₄), iron(II) sulfate, manganese(II) sulfate, manganese(II) chloride, cobalt(II) chloride, cerium sulfate, cerium chloride, vanadium sulfate, manganese dioxide (MnO₂).

It is also preferred according to the invention to use “complexing agents”. Complexing agents are substances which are capable of complexing metal ions. Preferred complexing agents are “chelate” complexing agents, namely substances which form cyclic compounds with metal ions, one individual ligand having more than one coordination site on a central atom, i.e. being at least “bidentate”. In this case, extended compounds are thus normally closed into rings by complexation via an ion. The number of bound ligands depends on the coordination number of the central ion.

Conventional chelate complexing agents which are preferred for the purposes of the present invention are for example polyoxycarboxylic acids, polyamines, ethylenediaminetetraacetic acid (EDTA), nitrilotriacetic acid (NTA) and hydroxyethanediphosphonic acids or the alkali metal salts thereof. Complex-forming polymers, thus polymers which, either in the main chain itself or laterally thereto, bear functional groups which are capable of acting as ligands and react with suitable metal atoms, generally resulting in the formation of chelate complexes, may also be used according to the invention. The polymer-bound ligands of the resultant metal complexes may here originate from just one macromolecule or alternatively belong to different polymer chains. The latter results in crosslinking of the material, providing the complexing polymers had not already previously been crosslinked via covalent bonds.

Complexing groups (ligands) of conventional complex-forming polymers are iminodiacetic acid, hydroxyquinoline, thiourea, guanidine, dithiocarbamate, hydroxamic acid, amide oxime, aminophosphoric acid, (cyclic) polyamino, mercapto, 1,3-dicarbonyl and crown ether residues, some exhibiting highly specific activities towards ions of different metals. Base polymers for many also commercially significant complex-forming polymers are polystyrene, polyacrylates, polyacrylonitriles, polyvinyl alcohols, polyvinylpyridines and polyethyleneimines. Natural polymers such as cellulose, starch or chitin are also complex-forming polymers. These may furthermore be provided with further ligand functionalities by polymer-analogous transformations.

It is particularly preferred for the purposes of the present invention to use one or more chelate complexing agents from the groups of
(i) polycarboxylic acids in which the total of carboxyl and optionally hydroxy groups amounts to at least 5,
(ii) nitrogenous mono- or polycarboxylic acids,
(iii) geminal diphosphonic acids,
(iv) aminophosphonic acids,
(v) phosphonopolycarboxylic acids,
(vi) cyclodextrins.

Any prior art complexing agents may be used for the purposes of the present invention. These may belong to different chemical groups. The following are preferably used individually or in combination with one another:
a) polycarboxylic acids in which the total of carboxyl and optionally hydroxy groups amounts to at least 5, such as gluconic acid,
b) nitrogenous mono- or polycarboxylic acids such as ethylene-diaminetetraacetic acid (EDTA), N-hydroxyethylethylenediaminetriacetic acid, diethylenetriaminepentaacetic acid, hydroxyethyliminodiacetic acid, nitridodiacetic acid-3-propionic acid, isoserinediacetic acid, N,N-di-(β-hydroxyethyl)glycine, N-(1,2-dicarboxy-2-hydroxyethyl)-glycine, N-(1,2-dicarboxy-2-hydroxyethyl)aspartic acid or nitrilotriacetic acid (NTA),
c) geminal diphosphonic acids such as 1-hydroxyethane-1,1-diphosphonic acid (HEDP), the higher homologs thereof with up to 8 carbon atoms as well as derivatives thereof containing hydroxy or amino groups and 1-aminoethane-1,1-diphosphonic acid, the higher homologs thereof with up to 8 carbon atoms as well as derivatives thereof containing hydroxy or amino groups,
d) aminophosphonic acids such as ethylenediaminetetra(methylene-phosphonic acid), diethylenetriaminepenta(methylenephosphonic acid) or nitrilotri(methylenephosphonic acid),
e) phosphonopolycarboxylic acids such as 2-phosphonobutane-1,2,4-tricarboxylic acid as well as
f) cyclodextrins.

For the purpose of the present patent application, polycarboxylic acids a) are taken to mean carboxylic acids, including monocarboxylic acids, in which the sum of carboxyl and hydroxy groups contained in the molecule amounts to at least 5. Complexing agents from the group of nitrogenous polycarboxylic acids, in particular EDTA, are preferred. At the alkaline pH values of the treatment solutions which are required according to the invention, these complexing agents are present at least in part as anions. It is immaterial whether they are introduced in the form of acids or in the form of salts. In the case of use as salts, alkali metal, ammonium or alkylammonium salts, in particular sodium salts, are preferred.

Polymeric aminodicarboxylic acids, the salts thereof or the precursor substances thereof may likewise be mentioned as further preferred complexing agents. Polyaspartic acids or the salts and derivatives thereof, which in addition to cobuilder properties also exhibit a bleach stabilizing action, are particularly preferred.

Further suitable complexing agents are polyacetals which may be obtained by reacting dialdehydes with polyolcarboxylic acids which comprise 5 to 7 C atoms and at least 3 hydroxy groups. Preferred polyacetals are obtained from dialdehydes such as glyoxal, glutaraldehyde, terephthalaldehyde as well as mixtures thereof and from polyolcarboxylic acids such as gluconic acid and/or glucoheptonic acid.

Phosphonates are another class of substances with complex-forming properties. These in particular comprise hydroxyalkane- or amino-alkanephosphonates. Among hydroxyalkanephosphonates, 1-hydroxyethane-1,1-diphosphonate (HEDP) is of particular significance. It is preferably used as a sodium salt, the disodium salt exhibiting a neutral reaction and the tetrasodium salt an alkaline (pH 9) reaction. Aminoalkanephosphonates which may preferably be considered are ethylenediaminetetramethylenephosphonate (EDTMP), diethylenetriaminepentamethylenephosphonate (DTPMP) as well as the higher homologs thereof. They are preferably used in the form of the sodium salts which exhibit a neutral reaction, for example as the hexasodium salt of EDTMP or as the hepta- and octasodium salt of DTPMP. HEDP is here preferably used as a complexing agent from the class of phosphonates. Aminoalkanephosphonates furthermore exhibit a pronounced heavy metal binding capacity. It may accordingly be preferred, especially if the agents also contain bleach, to use aminoalkanephosphonates, in particular DTPMP, or mixtures of the stated phosphonates. These substances are described below.

Complexing agents which are preferred according to the invention are phosphonates, preferably hydroxyalkane- or. aminoalkanephosphonates and in particular 1-hydroxyethane-1,1-diphosphonate (HEDP) or the di- or tetrasodium salt thereof and/or ethylenediaminetetramethylenephosphonate (EDTMP) or the hexasodium salt thereof and/or diethylene-triaminepentamethylenephosphonate (DTPMP) or the hepta- or octasodium salt thereof.

Agents which are particularly preferred according to the invention contain one or more substances from the group
(a) nitrilotriacetic acid (NTA),
(b) diethylenetriaminepentaacetic acid (DTPA),
(c) ethylenediaminedisuccinic acid (EDDS),
(d) ethylenediaminediglutaric acid (EDGA),
(e) 2-hydroxypropylenediaminedisuccinic acid (HPDS),
(f) glycineamide-N,N′-disuccinic acid (GADS),
(g) ethylenediamine-N,N′-diglutaric acid (EDDG),
(h) 2-hydroxypropylenediamine-N,N′-disuccinic acid (HPDDS),
(i) ethylenediaminetetraacetic acid (EDTA),
(j) ethylenedicysteic acid (EDC),
(k) diaminoalkyldi(sulfosuccinic acid) (DDS),
(l) ethylenediamine-N,N′-bis(ortho-hydroxyphenylacetic acid (EDDHA),
(m) N-2-hydroxyethyl-N,N-diacetic acid,
(n) glyceryliminodiacetic acid,
(o) iminodiacetic acid-N-2-hydroxypropylsulfonic acid,
(p) aspartic acid-N-carboxymethyl-N-2,5-hydroxypropyl-3-sulfonic acid,
(q) 8-alanine-N,N′-diacetic acid,
(r) aspartic acid-N,N′-diacetic acid,
(s) aspartic acid-N-monoacetic acid,
(t) dipicolinic acid,
(u) as well as the salts and/or derivatives thereof.

Some representatives from the above-stated groups of substances are particularly preferred for the purposes of the present invention. Agents for dyeing and/or lightening keratin fibers which are particularly preferred according to the invention additionally contain one or more chelate complexing agents from the groups of
(i) polycarboxylic acids in which the total of carboxyl and optionally hydroxy groups amounts to at least 5,
(ii) nitrogenous mono- or polycarboxylic acids,
(iii) geminal diphosphonic acids,
(iv) aminophosphonic acids,
(v) phosphonopolycarboxylic acids,
(vi) cyclodextrins,
   preferred agents containing phosphonates, preferably hydroxyalkane- or aminoalkanephosphonates and in particular 1-hydroxyethane-1,1-diphosphonate (HEDP) or the di- or tetrasodium salt thereof and/or ethylene-diaminetetramethylenephosphonate (EDTMP) or the hexasodium salt thereof and/or diethylenetriaminepentamethylenephosphonate (DTPMP) or the hepta- or octasodium salt thereof.

Preferred agents according to the invention are formulated with a low or zero water content. Agents which are particularly preferred according to the invention are characterized in that they contain less than 5 wt. %, preferably less than 2 wt. %, particularly preferably less than 1 wt. % and in particular less than 0.5 wt. % water, preferred agents being anhydrous. The water content of the agents may for example be determined by means of Karl Fischer titration.

The present invention secondly provides a method for lightening keratin fibers, in particular human hair, in which
if desired a pretreatment agent M1 is applied onto the fibers, then
an agent M2 is used on the fibers, a further agent M3 if desired being added to the agent M2 prior to use,
this agent M2 is rinsed off the fibers after a period of 5-30 minutes
and after the treatment a post-treatment agent M4 is optionally applied onto the fibers and rinsed off again after an exposure time of a few minutes,
   at least one of agents M1, M2 or M3 being an agent according to the invention.

The agents according to the invention may thus be formulated and correspondingly used as single component agents (dye and lightening agent M2 or post-treatment agent M4), as two-component agents (M2+M3) or as three-component agents (M2+M3+M4). Separation into multicomponent systems may in particular be considered where incompatibilities of the ingredients are to be expected or feared; in such systems, the agent for use is produced by the consumer directly before application by mixing the components.

A dyeing and lightening method in which the lightening cream and the oxidizing agent are initially separate is here preferred. The present invention accordingly also provides a method for dyeing and lightening human hair, in which a water-based composition containing hydrogen peroxide is mixed with an agent according to the invention to form a homogeneous composition and the latter is applied onto the hair.

In preferred methods according to the invention of this kind, the water-based composition contains, relative to the weight thereof, 1 to 20 wt. %, preferably 2 to 10 wt. % and in particular 3 to 6 wt. % hydrogen peroxide, calculated as 100% H₂O₂.

Further preferred methods according to the invention of this kind are characterized in that the water-based composition containing hydrogen peroxide is mixed with an agent according to the invention in a weight ratio of 1:5 to 10:1, preferably of 1:2 to 5:1 and in particular of 1:2 to 2:1 to form a homogeneous composition and the latter is applied onto the hair.

Alternatively, as mentioned above, a three-component system may also be used. The present invention accordingly also provides a method for dyeing and lightening human hair, in which a water-based composition containing hydrogen peroxide is mixed with a further agent preferably containing at least one alkalinity donor and/or direct hair dye and/or at least one oxidation dye precursor, and an agent according to the invention to form a homogeneous composition and the latter is applied onto the hair.

The above statements regarding the agents according to the invention apply mutatis mutandis with regard to further preferred embodiments of the methods according to the invention.

The present invention also provides the use of aldehydes of the formula (I):

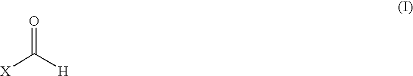

in which

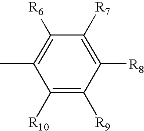

X denotes —CH(R²)—SO₂—Y—R¹, —CR³R⁴R⁵,
Y denotes —CH(CHO)— or —CH₂— or a chemical bond
R¹, R², R³, R⁴, R⁵, R⁶, R⁷, R⁸, R⁹, R¹⁰ mutually independently denote —H or —CN or —F or —Cl, or —Br, or —I or —CHO or —NH₂ or —NO₂ or —CF₃ or —CCl₃ or —CF₂CF₃ or —CCl₂CCl₃ or an optionally substituted (C₁-C₆) alkyl group or a hydroxyalkyl group or a polyhydroxyalkyl group or an optionally substituted (C₁-C₆) alkylene group,
for
vi. increasing lightening or blonding power and/or
vii. increasing the skin compatibility of lightening or blonding agents.

The explanations relating to preferred agents apply mutatis mutandis with regard to preferred uses according to the invention.

### EXAMPLES

The investigations were performed in aqueous solutions. Strands of dark blond hair (code: Kerling 7/0) weighing approx. 0.5 g had 15 times the quantity of active ingredient solution poured over them in beakers and were blonded for 30 minutes at 32° C. The pH value was adjusted to approx. 10.0 or 8.0.

The concentrations of hydrogen peroxide were in each case 6.0%, while the concentrations of acetaldehyde, chloral hydrate and benzaldehyde may be found in the table. The solutions were additionally stabilized with 1.5% Turpinal (60% solution of hydroxyethanediphosphonic acid) and 0.1% dipicolinic acid.

### Colorimetric Results:

| Test | L | a | b | dE |
|---|---|---|---|---|
| Starting hair, dark blond | 31.17 | 6.97 | 13.66 | |
| Dark blond + hydrogen | 41.37 | 9.14 | 21.88 | 13.28 |
| peroxide | | | | |
| pH 10 | | | | |
| Dark blond + hydrogen | 43.89 | 9.72 | 24.19 | 16.74 |
| peroxide + 5% acetaldehyde | | | | |
| pH 10 | | | | |
| Dark blond + hydrogen | 46.41 | 9.38 | 24.66 | 18.95 |
| peroxide + 3.5% chloral | | | | |
| hydrate | | | | |
| pH 10 | | | | |
| Dark blond + hydrogen | 49.35 | 9.04 | 25.45 | 21.77 |
| peroxide + 5.0% chloral | | | | |
| hydrate | | | | |
| pH 10 | | | | |
| Dark blond + hydrogen | 44.52 | 9.08 | 22.91 | 16.38 |
| peroxide + 5.0% benzaldehyde | | | | |
| pH 10 | | | | |
| Dark blond + hydrogen | 33.26 | 7.69 | 15.60 | 2.94 |
| peroxide | | | | |
| pH 8 | | | | |
| Dark blond + hydrogen | 37.33 | 8.65 | 19.02 | 8.34 |
| peroxide + 5% acetaldehyde | | | | |
| pH 8 | | | | |
| Dark blond + hydrogen | 36.5 | 8.28 | 18.12 | 7.07 |
| peroxide + 5.0% chloral | | | | |
| hydrate | | | | |
| pH 8 | | | | |
| Dark blond + hydrogen | 35.19 | 8.10 | 16.98 | 5.33 |
| peroxide + 5.0% benzaldehyde | | | | |
| pH 8 | | | | |

The measured values show that adding aldehydes to the hydrogen peroxide solutions brings about an improvement in lightening.

1-18. (canceled)

19. An agent for dyeing and/or lightening keratin fibers, in particular human hair, containing, relative to the weight thereof, 0.001 to 15 wt. % of at least one aldehyde of the formula (1): 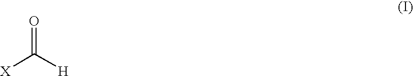 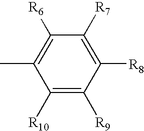
wherein X represents —CH(R²)—SO₂—Y—R¹, —CR³R⁴R⁵, or
wherein Y represents —CH(CHO)— or —CH₂— or a chemical bond, and
wherein each of R¹, R², R³, R⁴, R⁵, R⁶, R⁷, R⁸, R⁹, and R¹⁰ independently represents —H or —CN or —F or —Cl or —Br or —I or —CHO or —NH₂ or —NO₂ or —CF₃ or —CCl₃ or —CF₂CF₃ or —CCl₂CCl₃ or an optionally substituted (C₁-C₆) alkyl group or a hydroxyalkyl group or a polyhydroxyalkyl group or an optionally substituted (C₁-C₆) alkylene group,
and wherein the agent contains no oxidation dye precursors of developer and coupler type.

20. The agent for dyeing and/or lightening keratin fibers according to claim 19, wherein it contains one or more aldehydes from the group acetaldehyde, trichloroacetaldehyde, trifluoroacetaldehyde, nitroacetaldehyde, methylsulfonylacetaldehyde or benzaldehyde.

21. The agent for dyeing and/or lightening keratin fibers according to claim 19, wherein it contains the aldehyde(s) in quantities of 0.1 to 12.5 wt. %, preferably of 0.25 to 10 wt. %, particularly preferably of 0.5 to 7.5 wt. % and in particular of 1 to 5 wt. %, in each case relative to the entire agent.

22. The agent for dyeing and/or lightening keratin fibers according to claim 19, wherein it contains 0.5 to 15 wt. %, preferably 1 to 12.5 wt. %, particularly preferably 2.5 to 10 wt. % and in particular 3 to 6 wt. % of hydrogen peroxide (calculated as 100% H₂O₂).

23. The agent for dyeing and/or lightening keratin fibers according to claim 19, wherein, relative to the weight thereof, it additionally contains 0.001 to 5 wt. % of one or more direct dyes, preferred agents containing at least one direct dye which is selected from nitrophenylenediamines, nitroaminophenols, azo dyes, anthraquinones or indophenols, preferably from the group of dyes known by the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, Acid Black 52, Basic Yellow 87, Basic Orange 31 and Basic Red 51 as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2-hydroxyethyl)amino-4,6-dinitrophenol, 1-(2′-hydroxy-ethyl)amino-4-methyl-2-nitrobenzene, 1-amino-4-(2-hydroxyethyl)-amino-5-chloro-2-nitro-benzene, 4-amino-3-nitrophenol, 1-(2-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid and 2-chloro-6-ethylamino-1-hydroxy-4-nitrobenzene.

24. The agent for dyeing and/or lightening keratin fibers according to claim 19, wherein, relative to the weight thereof, it contains 1 to 20 wt. % of at least one imidazole compound according to the formula (VI) and/or the physiologically acceptable salts thereof 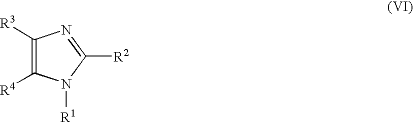
wherein R¹ represents a hydrogen atom, an optionally substituted aryl group, or a (C₁-C₆) alkyl group, R² represents hydrogen atom, a carboxaldehyde group, a (C₁-C₆) alkyl group or a nitro group, R³ represents a hydrogen atom, a carboxy-(C₁-C₆)-alkyl group, an amino-(C₁-C₆)-alkyl group, a carboxyl group, a carboxaldehyde group, a (C₁-C₆) alkyl group, a nitro group, a 2-amino-3-hydroxypropyl group or a —CH₂—CH(NH₂)—COON group, and R⁴ represents a hydrogen atom, a carboxaldehyde group or a carboxyl group.

25. The agent for dyeing and/or lightening keratin fibers according to claim 24, wherein the imidazole compound according to the formula (VI) and/or the physiologically acceptable salts thereof is/are selected from histamine, D-histidine, L-histidine, DL-histidine, D-histidinol, L-histidinol, DL-histidinol, imidazole, imidazole-4-acetic acid, imidazole-4-carboxylic acid, imidazole-4,5-dicarboxylic acid, imidazole-2-carboxaldehyde, imidazole-4-carboxaldehyde, imidazole-5-carboxaldehyde, 2-nitroimidazole, 4-nitroimidazole, 4-methylimidazole-5-carboxaldehyde, N-methylimidazole-2-carboxaldehyde, 4-methylimidazole, 2-methylimidazole, N-methylimidazole, N-(4-aminophenyl)-imidazole, as well as the physiologically acceptable salts of the above-stated compounds.

26. The agent for dyeing and/or lightening keratin fibers according to claim 19, wherein it additionally contains 0.01 to 2 wt. % of at least one solid peroxo compound which is selected from ammonium and alkali metal persulfates and peroxydisulfates, preferred agents containing peroxydisulfates which are preferably selected from sodium peroxydisulfate and/or potassium peroxydisulfate and/or ammonium peroxydisulfate, and particularly preferred agents containing at least two different peroxydisulfates.

27. The agent for dyeing and/or lightening keratin fibers according to claim 19, wherein it additionally contains 0.1 to 25 wt. %, preferably 0.5 to 20 wt. %, particularly preferably 1 to 15 wt. % an in particular 1.5 to 10 wt. % of at least one substance from the groups of
i. carbonates and/or
ii. monoalkyl carbonates (carbonic acid monoesters) and/or
iii. carbonic acid monoamides and/or
iv. silyl carbonates and/or
v. silyl carbamates.

28. The agent for dyeing and/or lightening keratin fibers according to claim 19 wherein it additionally contains
0.05 to 5 wt. % of at least one cyanate of the formula
M⁺[OCN]⁻
in which M⁺ denotes K⁺, Na⁺, Li⁺ or NH₄⁺ or ½Ca²⁺, ½Mg²⁺ or ½Zn²⁺ and/or
0.05 to 5 wt. % of at least one compound of the formula
H₂N—C(O)-L
in which L denotes a group displaceable by the anion ⁻OOH.

29. The agent for dyeing and/or lightening keratin fibers according to claim 28, wherein it contains 0.075 to 4 wt. %, preferably 0.1 to 2.5 wt. % and in particular 0.2 to 1 wt. % sodium cyanate and/or potassium cyanate and/or ammonium cyanate, sodium cyanate being particularly preferred.

30. The agent for dyeing and/or lightening keratin fibers according to claim 28, wherein it contains 0.075 to 4 wt. %, preferably 0.1 to 3.5 wt. % and in particular 0.2 to 3 wt. % of at least one compound of the formula
H₂N—C(O)-L
in which L is selected from —S—CH₂—COOH, —S—(CH₂)₃SO₃H, —S—(CH₂)₂—NH₂, —OCH₃, —O—(C₆H₄)—SO₃M, -Q⁺, —NH—OH, —O—C(O)—R¹, —O—C(O)—H, —O—CH(OH)₂, —SO₃M, —PH(O)OH, —P(O)(OH)₂, —(NH-glucosamine), —NH—C(O)—NH₂, —NH—NH₂, —NH—CN, —P(OH)(O)—C(O)NH₂, —SCN, —OCN, —S—C(NH)NH₂, 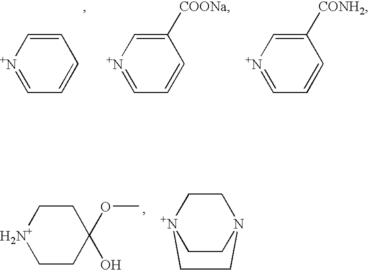
and M denotes —H, Na, K, NH₄, Q is selected from the quaternary ammonium residues formed from tertiary amines, heteroaryl residues or nonaromatic heterocycles and R¹ denotes —H, —CH₃, —CH₂CH₃, —(CH₂)₂CH₃, —CH(CH₃)₂ or another alkyl group.

31. The agent for dyeing and/or lightening keratin fibers according to claim 19, wherein it additionally contains Cu, Fe, Mn, Co, Ce, V, Ru ions or complexes of these ions, preferred agents containing 0.0001 to 2.5 wt. %, preferably 0.001 to 1 wt. % of at least one compound from the group copper chloride (CuCl₂), copper sulfate (CuSO₄), iron(II) sulfate, manganese(II) sulfate, manganese(II) chloride, cobalt(II) chloride, cerium sulfate, cerium chloride, vanadium sulfate, manganese dioxide (MnO₂).

32. A method for lightening keratin fibers, in particular human hair, wherein
if desired a pretreatment agent M1 is applied onto the fibers, then
an agent M2 is used on the fibers, a further agent M3 if desired being added to the agent M2 prior to use,
this agent M2 is rinsed off the fibers after a period of 5-30 minutes
and after the treatment a post-treatment agent M4 is optionally applied onto the fibers and rinsed off again after an exposure time of a few minutes,
wherein at least one of agents M1, M2 or M3 is an agent according to claim 19.

33. A method of increasing lightening or blonding power of a composition comprising providing an agent according to claim 19, and adding it to the composition.

34. A method of increasing skin compatibility of a lightening or blonding composition comprising providing an agent according to claim 19, and adding it to the composition.
